(19) United States
(12) Patent Application Publication
(10) Pub. No.: US 2013/0108670 A1
(43) Pub. Date: May 2, 2013

(54) COMPOSITIONS AND METHODS FOR TREATING FULL THICKNESS BURN INJURIES

(71) Applicant: BioMimetic Therapeutics, Inc., Franklin, TN (US)

(72) Inventors: Samuel Eugene Lynch, Franklin, TN (US); Jeffrey S. Guy, Franklin, TN (US)

(73) Assignee: BIOMIMETIC THERAPEUTICS, INC., Franklin, TN (US)

(21) Appl. No.: 13/658,922

(22) Filed: Oct. 24, 2012

Related U.S. Application Data
(60) Provisional application No. 61/551,179, filed on Oct. 25, 2011.
(60) Provisional application No. 61/551,202, filed on Oct. 25, 2011.

(51) Int. Cl. A61K 38/18; A61K 9/00

(52) U.S. Cl. CPC A61K 38/1858; A61K 9/00
USPC 424/400; 514/8.2; 514/2.3; 514/8.1

(57) ABSTRACT

The present invention provides compositions and methods for treating full thickness burn injuries. The present invention provides compositions and methods for promoting healing and regeneration of impaired or damaged tissue at a site of such a full thickness burn injury, as well as promoting vascularization and angiogenesis in regenerating tissue at such sites.

### CROSS-REFERENCE TO RELATED APPLICATIONS

This application claims the benefit of priority to U.S. Provisional Application Nos. 61/551,179, filed on Oct. 25, 2011, and 61/551,202, also filed on Oct. 25, 2011, both of which are hereby incorporated by reference in their entirety.

### FIELD OF THE INVENTION

The present invention relates to compositions and methods useful for treating full thickness burn injuries, and for promoting healing and regeneration of tissue that is damaged or impaired at the site of a full thickness burn injury. The invention also relates to compositions and methods for promoting vascularization and angiogenesis of regenerating tissue at such sites, and for promoting the growth of supportive tissues which support the survival of a skin flap or graft that is applied to such sites.

### BACKGROUND OF THE INVENTION

Burn injuries are among the oldest, most complex and painful injuries known. Dating to antiquity, humans have been battling the devastating effects of burns. The incidence of burn injuries is high, with 1.25 million such injuries occurring in the U.S alone, requiring 600,000 emergency room visits and 45,000 hospitalizations. Burn injuries are the second leading cause of accidental death in the United States.

The more severe burn injuries, for example, full thickness burn injuries, involve damage or impairment to all layers of the skin, as well as underlying subcutaneous tissue and deeper tissues, including bone, muscle, tendon, vascular tissue, and/or nerve tissue at the site of the burn. Treatment of, and recovery from, such injuries is often complex, lengthy, painful, and emotionally draining.

Current treatments for full thickness burn injuries typically involve one or more debridements and/or escharotomies at the site of the injury in order to remove severely damaged and/or necrotic or infected tissues. In cases in which there is damage or impairment to bone, decortications of the bone may also be required. Additionally, wound dressings and/or skin flaps or grafts are typically required to facilitate healing and closure of the injury site. Skin flaps or skin grafts, which are layers of skin with or without supporting vascular tissue, respectively, are typically taken from a suitable donor area of a patient and transplanted to a recipient area of damaged skin or to a site where an amputation has been performed.

However, in many cases, particularly where significant damage to deeper tissues such as muscle, tendon, and bone has occurred, there is often a lack of sufficient vascularized supporting tissue at, around or within the site of the injury to adequately support the viability of the skin flap or graft. Absent such supportive tissues (often termed a “wound bed”, “tissue bed”, or “vascularized tissue bed”), sufficient oxygen and nutrient supplies are not available to the skin flap or graft, and assimilation of the skin flap or graft is delayed or compromised, increasing the length of time for recovery, as well increasing the chance for infection to set in and/or worsen. Unfortunately, in many cases, inadequate supportive tissue may cause the partial or complete failure of a skin flap or graft to “take”. Additionally, persistent infection at the site may spread to surrounding tissues and/or enter the blood stream and lead to sepsis, septic shock, organ failure, and even death.

Thermal burns are by far the most common types of burns. Although the skin is usually the part of the body that is burned, the tissues under the skin can also be burned, and internal organs can be burned even when the skin is not. For example, drinking a very hot liquid or caustic substance such as acid can burn the esophagus and stomach. Inhaling smoke or hot air from a fire burn the lungs. When tissues are damaged by a burn, fluid may leak from blood vessels (capillary permeability), causing swelling or edema. In an extensive burn, loss of a large amount of fluid from abnormally leaky blood vessels can cause shock. In shock, blood pressure decreases so much that too little blood flows to the brain and other vital organs.

Electrical burns may be caused by a temperature of more than 9,000° F., generated by an electric current when it passes from the electrical source to the body. This type of burn, sometimes called an electrical arc burn, usually completely destroys and chars the skin at the current's point of entry into the body. Because the resistance (the body's ability to stop or slow the current's flow) is high where the skin touches the current's source, much of the electrical energy is converted into heat, thus burning the surface. Most electrical burns also severely damage the tissues under the skin, including fascia, muscle, tendon, nerve tissue, and bone. These burns vary in size and depth and may affect an area much larger than that indicated by the area of injured skin. Large electrical shocks can paralyze breathing and disturb heart rhythm, causing dangerously irregular heartbeats.

Chemical burns can be caused by various irritants and poisons, including strong acids and alkalis, phenols and cresols (organic solvents), mustard gas, and phosphorus. Chemical burns can cause tissue death that can slowly spread for hours after the burn.

Radiation burns can be caused by nuclear weapons, nuclear accidents, laboratory exposure, accidents during X-ray radiation chemotherapy, and over-exposure to sun. Radiation burns can cause inflammation, edema, ulcerations, damage to underlying endothelium and other cell types, as well as mutagenesis resulting in cancer, especially hematologic malignancies.

After suffering a burn injury, the affected individual can experience severe protein, muscle, and fat wasting in the area of the burn. Indeed, loss of up to 20% of body protein may occur in the first two weeks following a third degree or deep tissue, e.g., a full thickness, burn injury. Increased oxygen consumption, metabolic rate, urinary nitrogen excretion, fat breakdown and steady erosion of body mass are all directly related to burn size. A return to normal levels as the burn wound heals gradually restores chemical balance, temperature and pH.

As mentioned above, the incidence of developing an infection at the site of the injury is high. As a result, local administration of antibiotics, is common in these burn injury cases. However, the prevalence of devascularization and/or hypoxia in damaged and/or necrotic tissue at such burn injury sites often promotes an environment that is both nutritive and hospitable for microbial growth and frustrates the delivery of effective amounts of antimicrobial agents, such as antibiotics, to the site of the infection. As a result, local infections at the site of such injuries are often prone to developing into systemic infections, which often require systemic administration of antibiotics. Thus, the timeframe for wound healing, including the establishment of a vascularized bed of supportive tissue to facilitate graft assimilation and wound closure becomes critical, as protracted timeframes increase the chance that infection will develop, persist, and increase in severity.

In view of the severity and complexity of full thickness burn injuries and associated risks of persistent local and systemic microbial infection and sepsis, and the shortcomings of current treatment methods to address such issues, there is a need to provide compositions and methods that promote and improve the healing, regeneration, and growth of tissue that has been impaired or damaged at the site of these injuries. Such compositions and methods would not only increase the chances of salvaging or repairing damaged structures and functionalities, but would also lessen the time to recovery, decrease the chance for infection to develop and persist, and ultimately, improve patient outcomes.

### BRIEF SUMMARY

The present invention provides compositions and methods for treating a site of full thickness burn injuries, wherein the methods comprise applying to the site a composition comprising a biocompatible matrix and an effective amount of platelet-derived growth factor (PDGF) disposed therein. In other embodiments, the method comprises debriding at least a portion of the site; decorticating at least a portion of impaired or damaged bone at the site; performing at least one intramarrow bone penetration at the site to promote egress of marrow components from marrow into the site: and applying to the site a composition comprising a biocompatible matrix and an effective amount of platelet-derived growth factor (PDGF) disposed therein. The full thickness burn injury may comprise a third degree burn, a fourth degree burn, a chemical burn, a thermal burn, radiation burn or an electrical burn. Also provided are compositions and methods for: promoting healing or regeneration of damaged or impaired tissue; promoting vascularization in regenerating tissue; promoting angiogenesis in regenerating tissue; promoting the growth of a vascularized tissue bed; and promoting the growth of supportive tissue; at a site of a full thickness burn injury. In certain embodiments, the tissue that is impaired or damaged and/or regenerating is selected from the group consisting of bone, periosteum, tendon, muscle, fascia, nerve tissue, dermal tissue, vascular tissue, and combinations thereof. In some embodiments the tissue comprises periosteal tissue. Moreover, the impaired or damaged tissue may be or may become necrotic prior to employing the disclosed methods.

In certain embodiments, the methods promote egress of at least one marrow component selected from the group consisting of periosteogeneic cells, angiogenic cells, stromal cells, mesenchymal cells, osteoprogenitor cells, osteoblasts, osteoclasts, platelets, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α(TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-BB, PDGF-CC, PDGF-DD, an osteogenin, and combinations thereof, into the site. The egress of such components promotes infiltration of such components into the biocompatible matrix.

In certain other embodiments the subject develops or is at risk of developing an infection at the site of the full thickness burn injury. Moreover, in some embodiments, the subject develops or is at risk of developing at least one of the following conditions: sepsis, septic shock, necrotizing fasciitis, gangrene, or osteomyelitis.

In certain embodiments, the disclosed methods further comprise disposing in the biocompatible matrix one or more subsequent effective amounts of PDGF. The one or more subsequent effective amounts of PDGF may be disposed in the biocompatible matrix within one approximately hour, from approximately one hour to approximately 12 hours, from approximately 12 hours to approximately 24, from approximately 24 hours to approximately seven days, from approximately one week to approximately two weeks, from approximately two weeks to approximately one month, from approximately one month to approximately two months, from approximately two months to approximately six months, from approximately six months to approximately one year, or combinations thereof, after performing the initial applying step. In certain embodiments, the subsequent amount of PDGF is disposed in the matrix approximately one week after performing the initial applying step. In certain embodiments one or more subsequent effective amounts of PDGF are periodically disposed in the biocompatible matrix that has been previously applied to the site of the full thickness burn injury. Additionally, the intervals between such periodic applications of PDGF may be any interval ranging from approximately 12 hours to approximately 180 days, and preferably at an interval of approximately 3, 5, 7, 10, 14, 15, 21, 28, 30, 60, 90 or 180 days, or any combinations thereof. The intervals between applications may or may not be equal intervals, and may be shorter during the earlier phases of the treatment. The periodic applications of PDGF may be applied over a period ranging from approximately one day to approximately one year, and preferably over a period of at least approximately one week, at least approximately one month, at least approximately two months, at least approximately three months, at least approximately four months, at least approximately five months, at least approximately six months, at least approximately nine months or at least approximately one year after performing the initial applying step.

In other embodiments, the methods further comprise applying one or more subsequent compositions comprising a biocompatible matrix and an effective amount of PDGF disposed therein to the site of full thickness burn injury. In accordance with this method, previously applied matrix may be removed prior to applying the subsequent composition. The one or more subsequent compositions may be applied to the site within approximately one hour, from approximately one hour to approximately 12 hours, from approximately 12 hours to approximately 24, from approximately 24 hours to approximately seven days, from approximately one week to approximately two weeks, from approximately two weeks to approximately one month, from approximately one month to approximately two months, from approximately two months to approximately six months, from approximately six months to approximately one year, or combinations thereof, after performing the initial applying step. In certain embodiments, such a subsequent composition is applied to the site approximately one week after performing the initial applying step. In certain embodiments, one or more subsequent compositions are periodically applied to the site. Additionally the intervals between such periodic applications of subsequent compositions may be any interval ranging from approximately 12 hours to approximately 180 days, and preferably at an interval of approximately 3, 5, 7, 10, 14, 15, 21, 28, 30, 60, 90 or 180 days, or any combinations thereof. The intervals between applications may or may not be equal intervals, and may be shorter during the earlier phases of the treatment. The periodic applications of subsequent compositions may be applied over a period ranging from approximately one day to approximately one year, and preferably over a period of at least approximately one week, at least approximately one month, at least approximately two months, at least approximately three months, at least approximately four months, at least approximately five months, at least approximately six months, at least approximately nine months, or at least approximately one year after performing the initial applying step.

In certain embodiments, the methods further comprise the steps of periodically retreating the site, wherein said retreatment consists of either: (1) removing all or a portion of the previously applied biocompatible matrix and applying at least one subsequent composition comprising a biocompatible matrix and an effective amount of PDGF disposed therein to the site, (2) applying at least one subsequent composition comprising a biocompatible matrix and an effective amount of PDGF disposed therein to the site, or (3) applying an effective amount of PDGF to a previously applied biocompatible matrix. In such accordance with this embodiment, the periodic retreatment may alternate between applying a composition comprising a biocompatible matrix and an effective amount of PDGF disposed therein, and applying an effective amount of PDGF to a previously applied biocompatible matrix. The intervals between the periodic retreatments may be any interval ranging from approximately 12 hours to approximately 180 days, and preferably at an interval of approximately 3, 5, 7, 10, 14, 15, 21, 28, 30, 60, 90 or 180 days, or any combinations thereof. The intervals between applications may or may not be equal intervals, and may be shorter during the earlier phases of the treatment. The periodic retreatments may occur over a period ranging from approximately one day to approximately one year, and preferably over a period of at least approximately one week, at least approximately one month, at least approximately two months, at least approximately three months, at least approximately four months, at least approximately five months, at least approximately six months, at least approximately nine months, or at least approximately one year after performing the initial treatment.

In accordance with all of the embodiments disclose herein, all or a portion of an initially applied and/or previously applied biocompatible matrix may be removed from the site prior to applying a subsequent biocompatible matrix or compositions comprising a biocompatible matrix.

Accordingly, in certain embodiments, the method includes a first phase comprising the step of periodically retreating the site by applying a biocompatible matrix and an effective amount of PDGF disposed therein, either with or without removing all or a portion of the previously applied biocompatible matrix, and a second phase comprising the step of periodically retreating the site by applying an effective amount of PDGF to the previously applied biocompatible matrix, thereby allowing such matrix to act as a scaffold for cellular growth, and become resorbed by the patient's body. The retreatments may occur at intervals outlined above and over the periods of time outlined above. In accordance with this embodiment, the first phase may occur over a period of ranging from approximately one day to approximately three months.

In certain embodiments, the biocompatible matrix comprises a mesh, a gauze, a sponge, a monophasic plug, a biphasic plug, a paste, a putty, a wrap, a bandage, a patch, a mesh, or a pad.

In certain embodiments, the biocompatible matrix is resorbable, porous, and/or resorbable and porous.

The biocompatible matrix may comprise one or more of the following: proteins, polysaccharides, nucleic acids, carbohydrates, inorganic components or minerals, and synthetic polymers; or mixtures or combinations thereof. Moreover, the biocompatible matrix may comprise a polyurethane, a siloxane, a polysiloxane, a collagen, a glycosaminoglycan, oxidized regenerated cellulose (ORC), an ORC:collagen composite, an alginate, an alginate:collagen composite, a ethylene diamine tetraacetic acid (EDTA),a poly(lactic-co-glycolitic acid (PLGA), a carboxymethylcellulose, a granulated collagen-glycosaminoglycan composite, methylcellulose, hydroxypropyl methylcellulose, or hydroxyethyl cellulose alginic acid, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), polyvinyl alcohol), poly(vinylpyrrolidone), poly(ethyloxazoline), poly(ethylene oxide)-co-polypropylene oxide) block copolymers, poly(ethylene terephthalate)polyamidearabic gum, guar gum, xantham gum, gelatin, chitin, chitosan, chitosan acetate, chitosan lactate, chondroitin sulfate, N,O-carboxymethyl chitosan, a dextran, fibrin glue, glycerol, hyaluronic acid, sodium hyaluronate, a cellulose, a glucosamine, a proteoglycan, a starch, lactic acid, a pluronic, sodium glycerophosphate, glycogen, a keratin, a silk, one or more composites thereof, one or more mixtures thereof, or one or more combinations thereof.

The biocompatible matrix, in some embodiments, comprises calcium phosphate. In one embodiment, a calcium phosphate comprises a β-TCP. In one aspect, biocompatible matrices may include calcium phosphate particles or bone allograft such as demineralized freeze dried bone allograft (DFDBA) or particulate demineralized bone matrix (DBM). In another aspect, biocompatible matrices may include bone allograft such as DFDBA or DBM. In such embodiments, the biocompatible matrix optionally comprises interconnected pores.

The biocompatible matrix may comprise a collagen. In certain embodiments, the biocompatible matrix comprises a Type I collagen, a Type II collagen, a Type III collagen, a Type IV collagen, a Type V collagen, a Type VI collagen, a Type VII collagen, a Type VIII collagen, or combinations thereof. Moreover, the collagen comprise bovine collagen, human collagen, porcine collagen, equine collagen, avian collagen, or combinations thereof In certain embodiments, the collagen comprises bovine Type I collagen or human Type I collagen.

In certain embodiments, the biocompatible matrix comprises an in vitro-prepared tissue or an in vitro prepared dermal tissue as disclose in, e.g., U.S. patent application Ser. No. 13/042,295, published as US publication number U.S. 2011/0245929, the disclosures of which are herein incorporated by reference in their entireties. Preferred in vitro prepared tissues are those tissues that comprise viable fibroblasts or are synthesized by or derived from fibroblasts, as disclosed in, e.g., U.S. patent application Ser. No. 13/042,295, published as U.S. publication number U.S. 2011/0245929. In certain embodiments, biocompatible matrices disclosed in Ser. No. 13/042,295, published as U.S. publication number U.S. 2011/0245929, are applied, using the devices and methods disclosed therein for implanting in vitro-prepared tissues and in vitro-prepared dermal tissues, to a site of a full thickness burn injury.

In certain embodiments, biocompatible matrix comprises: an AUGMENT Rotator Cuff Graft or another biocompatible matrix as disclosed in, for example U.S. patent application Ser. No. 11/772, 646, published as US 2008/0027470, the disclosures of which are hereby incorporated by reference in their entirety for all purposes; COLLATAPE; COLLACOTE; SOLOSITE; an INTEGRA dermal regeneration template; an INTEGRA cross-linked bovine tendon and glycosaminoglycan meshed bilayer wound matrix; a HELISTAT absorbable collagen hemostatic sponge; a HELITENE absorbable collagen hemostatic agent; an INTEGRA flowable wound matrix; Matrix Collagen Particles™ wound dressing; Matrix Collagen Sponge™ wound dressing; OssiMend™ bone graft matrix mineral:collagen composite; OssiMend™ block bone graft matrix mineral:collagen composite; OssiMend™ putty bone graft matrix mineral:collagen composite; OssiPatch™ collagen bone healing protective sheet; NeuroMatrix™ collagen nerve conduit; Neuroflex™ flexible collagen nerve conduit; NeuroMend™ collagen wrap conduit; Collatene™ fibrillar collagen dental dressing; SynOss™ synthetic mineral; OASIS® wound matrix; BIOBLANKET surgical mesh; ZIMMER collagen repair patch; DeNovo NT Graft marketed by ZIMMER as disclosed in, e.g., U.S. Pat. No. 7,824,711, the disclosure of which is hereby incorporated by reference in its entirety for all purposes; DeNovo ET engineered tissue graft marketed by Zimmer; small intestinal submucosa (SIS) as disclosed in, e.g., U.S. Pat. No. 8,025,896, the disclosure of which is hereby incorporated by reference in its entirety for all purposes; a composite structure such as disclosed in, e.g., U.S. patent application Ser. No. 12/997,611 (published as US 20110091515) the disclosures of which are hereby incorporated in their entireties for all purposes; PROMOGRAN matrix wound dressing; PURAPLY collagen dressing; or FIBROCOL PLUS® collagen dressing.In other embodiments, the method further comprises applying a dressing, a skin flap, or a graft at the site after performing the applying step or the retreatment step, or after sufficient supportive tissues have formed. The method promotes the growth of supportive tissue that supports the survival of a skin flap or graft that is applied at the site, in certain embodiments. The dressing, skin flap, or graft may comprise one or more of the following: a cadaver-derived skin graft; an animal skin graft; a skin autograft; a skin allograft; a synthetic skin substitute; a dermal substitute; fibroblast-derived temporary skin substitute; an artificial skin; a reconstructive tissue matrix; an acellular tissue matrix; an acellular dermal matrix (ADM); a processed human tissue graft; a reconstructive tissue matrix; a bioengineered cell construct; a bioengineered collagen matrix; an engineered tissue graft; a cellular and collagen construct; a collagen dressing; a human bi-layered bio-engineered skin; a polyglactin mesh scaffold; a graft or composition for grafting as disclosed in, e.g., U.S. Pat. No. 6,979,670, the disclosure of which is hereby incorporated by reference in its entirety; a composite structure such as disclosed in, e.g., U.S. patent application Ser. No. 12/997,611 (published as US 20110091515) the disclosures of which are hereby incorporated in their entireties for all purposes; a dermal substitute; an acellular dermal matrix (ADM) as disclosed in, e.g., U.S. patent Ser. No. 7,972,631 and 7,358,284, the disclosures of which are hereby incorporated in their entireties for all purposes; or a polyglactin mesh scaffold.

In certain embodiments the dressing, skin flap, or graft comprises DERMAGRAFT human fibroblast-derived dermal substitute, TRANSCYTE human fibroblast-derived temporary skin substitute, ALLODERM acellular tissue matrix, DermaMatrix® dermal matrix, Tutoplast® processed human tissue graft, Strattice™ reconstructive tissue matrix, PURAPLY collagen dressing, INTEGRA Artificial Skin, DeNovo NT graft marketed by ZIMMER, DeNovo ET Engineered Tissue Graft marketed by Zimmer, BIOBRANE temporary composite wound dressing, APLIGRAF bioengineered cell construct, CELTX living cellular and collagen construct, FORTAFLEX bioengineered collagen matrix, FORTAGEN collagen construct, or VC101 human bi-layered bio-engineered skin In some embodiments, an effective amount of PDGF is disposed in the dressing, the skin flap, or the graft.

In certain embodiments, an antimicrobial agent, such as an antiseptic, an antibacterial agent an antibiotic, an iodine-containing agent, a peroxide-containing agent, a silver-containing agent, iodine, povidone-iodine, an iodide ion-containing agent, hydrogen peroxide, a peroxide ion-containing agent, or a silver ion-containing agent, chlorhexadine, and the like, is applied to the site of the burn injury. In other embodiments, the antimicrobial agent is disposed in the biocompatible matrix. In further embodiments, the antimicrobial agent is disposed in the dressing, the skin flap or the graft. The antimicrobial agent may be further selected from the group consisting of: mafenide-acetate, penicillin, ampicillin, penicillin G, clindamycin (Cleocin), Ceftriaxone (Rocephin), erythromycin, gentamicin (Garamycin), clindamycin (Cleocin), metronidazole (Flagyl), Ampicillin-sulbactam (Unasyn), ticarcillin-clavulanate potassium (Timentin), piperacillin-tazobactam (Zosyn), nafcillin (Unipen), Imipenem-cilastatin (Primaxin), a β-lactam, a β-lactamase inhibitor, antipseudomonal cephalosporin, ceftazidime (Fortaz), clindamycin, metronidazole, Vancomycin (Vancocin) an aminoglycoside, aztreonam (Azactam), amphotericin B (Abelcet), a third-generation cephalosporin, and combinations thereof.

In certain embodiments, such an antimicrobial agent is systemically administered to the patient, or such an antimicrobial agent is administered locally at the site of the burn injury, or a combination thereof. Moreover, such an antimicrobial agent may be disposed in the matrix, systemically administered to the patient, administered locally at the site of the burn injury, or a combination thereof, before, after, or concomitant to performing the applying step and/or the step of retreatment. In certain embodiments, such an antimicrobial agent may be disposed in the dressing, the skin flap, or the graft.

In certain embodiments, at least one further biological agent is administered to the subject. The at least one further biological agent may be selected from the group consisting of: an albumin, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-CC, PDGF-DD, an osteogenin, a protease inhibitor, a metalloproteinase inhibitor, a metalloproteinase-3 inhibitor, ethylenediaminetetraacetic acid (EDTA), ethylene glycol-bis(beta-aminoethylether)-N, N,N′,N′-tetraacetic acid (EGTA), aprotinin, and ε-aminocaproic acid (EACA).

In certain embodiments, PDGF comprises PDGF homodimers and heterodimers, including PDGF-AA, PDGF-BB, PDGF-AB, PDGF-CC, PDGF-DD, and mixtures and derivatives thereof. In some embodiments, PDGF is obtained from a natural source or a recombinant source. In certain embodiments, the natural source comprises blood, platelets, serum, platelet concentrate, platelet-rich plasma (PRP), platelet-poor plasma (PPP), platelet-free plasma, plasma, fresh frozen plasma (FFP), or bone marrow. In one embodiment, PDGF comprises PDGF-BB. In another embodiment PDGF comprises a recombinant human (rh) PDGF such as recombinant human PDGF-BB (rhPDGF-BB).

In other embodiments of the present invention, PDGF comprises PDGF fragments. In one embodiment rhPDGF-B comprises the following fragments: amino acid sequences 1-31, 1-32, 33-108, 33-109, and/or 1-108 of the entire B chain. The complete amino acid sequence (1-109) of the B chain of PDGF is provided in FIG. 15 of U.S. Pat. No. 5,516,896. It is to be understood that the rhPDGF compositions of the present invention may comprise a combination of intact rhPDGF-B (1-109) and fragments thereof. Other fragments of PDGF may be employed such as those disclosed in U.S. Pat. No. 5,516,896, the disclosure of which is hereby incorporated by reference in its entirety. In accordance with a preferred embodiment, the rhPDGF-BB comprises at least 65% of intact rhPDGF-B (1-109).

In certain embodiments, the PDGF is in a pharmaceutically acceptable liquid. Moreover, the PDGF may be at a concentration from about 0.01 mg/ml to about 10 mg/ml; from about 0.05 mg/ml to about 5 mg/ml; from 0.1 to about 1.0 mg/ml; from about 0.2 to about 0.75 mg/ml; from about 0.25 to about 0.6 mg/ml; from about 0.01 to about 0.5 mg/ml; from about 0.01 mg/ml to about 0.4 mg/ml; from about 0.01 mg/ml to about 0.3 mg/ml; from about 0.01 mg/ml to about 0.3 mg/ml; from about 0.01 mg/ml to about 0.2 mg/ml; from about 0.05 mg/ml to about 0.3 mg/ml; about 0.01 mg/ml; about 0.02 mg/ml; about 0.03 mg/ml; about 0.04 mg/ml; about 0.05 mg/ml; about 0.06 mg/ml; about 0.07 mg/ml; about 0.08 mg/ml; about 0.09 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.0 mg/ml; 0.1 mg/ml; 0.3 mg/ml; 1.0 mg/ml and combinations thereof.

In certain embodiments, the pharmaceutically acceptable liquid comprises a buffer solution. In some embodiments, the buffer solution comprises a carbonate, a phosphate, phosphate buffered saline, histidine, an acetate, sodium acetate, acetic acid, hydrochloric acid, an organic buffer, lysine, a Tris buffer, tris(hydroxymethyl)aminoethane), N-2-hydroxyethylpiperazine-N′-2-ethanesulfonic acid (HEPES), or 3-(N-morpholino) propanesulfonic acid (MOPS). In some embodiments, the buffer solution comprises sodium acetate at a molarity of 20 mM.

In certain embodiments, the methods increase the survival rate of a skin flap or a graft that is applied to the burn injury site. In some embodiments, the methods promote vascularization and tissue growth at the site such that the survival rate of the skin flap or the graft is increased.

In another aspect, the present invention provides a kit comprising a solution comprising PDGF in a first container and a second container comprising a biocompatible matrix. In some embodiments, the solution comprises a predetermined concentration of PDGF. The concentration of PDGF, in some embodiments, can be predetermined according to the nature of the burn injury site to which a composition according to the invention is to be applied. The kit may further comprise at least one further biological agent and/at least one further biocompatible matrix component as disclosed herein throughout. Moreover, the amount of biocompatible matrix provided by a kit can be dependent on the nature of the burn injury site to which a composition according to the invention is to be applied. In certain embodiments, the kit comprises at least one collagen, such as a Type I collagen, a Type II collagen, a Type III collagen, a Type IV collagen, a Type V collagen, a Type VI collagen, a Type VII collagen, or a Type VIII collagen. A syringe, in some embodiments, can facilitate disposition of the PDGF solution in the biocompatible matrix for application at a burn injury site. The kits may also comprise an in vitro-prepared tissue or in vitro-prepared dermal tissue as disclosed in, e.g., U.S. patent application Ser. No. 13/042,295, published as U.S. publication number U.S. 2011/0245929. The kit may also contain instructions for use.

A further object of the present invention is to provide a composition for use in the preparation of a medicament for performing the disclosed methods.

These and other embodiments of the present invention are described in greater detail in the detailed description which follows. These and other objects, features and advantages of the present invention will become apparent after a review of the following detailed description of the disclosed embodiments and claims.

### DETAILED DESCRIPTION

The present disclosure provides compositions and methods for treating a site of a burn injury, wherein the methods comprise applying to the site a composition comprising a biocompatible matrix and an effective amount of platelet-derived growth factor disposed therein. In particular embodiments, the present invention provides methods for treating the site of a full thickness burn injury. In more particular embodiments, the present disclosure provides compositions and methods for treating a site of a full thickness burn injury, wherein the methods comprise: debriding at least a portion of the site; decorticating at least a portion of impaired or damaged bone at the site; performing at least one intramarrow bone penetration at the site to promote egress of marrow components from marrow into the site: and applying to the site a composition comprising a biocompatible matrix and an effective amount of platelet-derived growth factor (PDGF) disposed therein. The full thickness burn injury may comprise a third degree burn, a fourth degree burn, a chemical burn, a thermal burn, radiation burn or an electrical burn. Also provided are compositions and methods for: promoting healing or regeneration of damaged or impaired tissue; promoting vascularization in regenerating tissue; promoting angiogenesis in regenerating tissue; promoting the growth of a vascularized tissue bed; and promoting the growth of supportive tissue; at a site of a full thickness burn injury. In certain embodiments, the methods described herein promote the infiltration of cells into the biocompatible matrix.

In certain embodiments, the tissue that is impaired or damaged and/or regenerating is selected from the group consisting of bone, periosteum, tendon, muscle, fascia, nerve tissue, dermal tissue, vascular tissue, and combinations thereof. In some embodiments the tissue comprises periosteal tissue. Moreover, the impaired or damaged tissue may be or may become necrotic prior to employing the disclosed methods. Thus, in certain embodiments, the site comprises necrotic tissue.

In certain embodiments, the present disclosure provides a method of promoting healing or regeneration of damaged or impaired tissue at a site of a full thickness burn injury in a subject in need thereof, the method comprising applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

The disclosure further provides, in other embodiments, a method of promoting vascularization in regenerating tissue at a site of a full thickness burn injury in a subject in need thereof, wherein the method comprises applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

In yet another embodiment, the disclosure provides a method of promoting angiogenesis in regenerating tissue at a site of a full thickness burn injury in a subject in need thereof, wherein the method comprises applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

In still a further embodiment, the disclosure provides a method of promoting growth of vascularized tissue bed at a site of a full thickness burn injury in a subject in need thereof, wherein the method comprises applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

The tissue in these aforementioned and other embodiments may be selected from the group consisting of bone, periosteum, tendon, muscle, fascia, nerve tissue, vascular tissue, and combinations thereof.

In particular embodiments, the tissue is impaired or damaged periosteal tissue. Thus, the disclosure provides a method of treating impaired or damaged periosteal tissue at a site of a full thickness burn injury in a subject in need thereof, wherein the method comprises applying a composition comprising a biocompatible matrix and an effective amount of platelet-derivedgrowth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

In another embodiment, the disclosure provides a method of promoting healing or regeneration of impaired or damaged periosteal tissue at a site of a full thickness burn injury in a subject in need thereof, wherein the method comprises applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

In yet another embodiment, the disclosure provides a method of promoting vascularization in regenerating periosteal tissue at a site of a full thickness burn injury in a subject in need thereof, the method comprising applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

Another embodiment provides a method of promoting angiogenesis in regenerating periosteal tissue at a site of a full thickness burn injury in a subject in need thereof, the method comprising applying a composition comprising a biocompatible matrix and an effective amount of platelet-derived growth factor (PDGF) disposed therein to the site. In a more particular embodiment, the method comprises: a) debriding at least a portion of the site; b) decorticating at least a portion of impaired, damaged or necrotic bone at the site; c) performing at least one intramarrow bone penetration at the site to promote egress of marrow components from marrow into the site; and d) applying a composition comprising a biocompatible matrix and an effective amount of platelet-derived growth factor (PDGF) disposed therein to the site.

As discussed above, the impaired or damaged tissue at the burn injury site may be or may become necrotic prior to employing the disclosed methods. Accordingly, in certain embodiments of the present disclosure, the site comprises necrotic tissue. In these embodiments, the method may further comprise debriding at least a portion of the site of impaired, damaged, or necrotic tissue prior to performing step (a).

Certain embodiments include performing at least one intramarrow bone penetration at the site. The intramarrow penetration may, in some embodiments, promotes egress of marrow components from marrow into the site. For example, the marrow components may comprise at least one of the following: periosteogeneic cells, angiogenic cells, stromal cells, mesenchymal cells, osteoprogenitor cells, osteoblasts, osteoclasts, platelets, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-BB, PDGF-CC, PDGF-DD, an osteogenin, an in vitro-prepared tissue, an in vitro-prepared dermal tissue, a biological component made by an in vitro-prepared tissue, a biological component derived from an in vitro-derived tissue, or combinations thereof.

In a more particular embodiment, the disclosure provides a method for treating a site of a full thickness burn injury in a subject in need thereof, the method comprising: (a) debriding at least a portion of impaired, damaged, or necrotic tissue at the site; (b) decorticating at least a portion of impaired, damaged or necrotic bone at the site; (d) performing at least one intramarrow bone penetration at the site to promote egress of marrow components from marrow into the site; (d) applying an initial treatment of a first composition to the site, wherein the first composition comprises a biocompatible matrix comprising collagen having incorporated therein a liquid comprising platelet-derived growth factor (PDGF) at a concentration in a range of about 0.05 mg/mL to about 10.0 mg/mL in a pharmaceutically acceptable liquid; and (e) applying one or more subsequent treatments of additional compositions to the site, wherein the additional compositions comprise either (i) a biocompatible matrix comprising collagen having incorporated therein a liquid comprising platelet-derived growth factor (PDGF) at a concentration in a range of about 0.05 mg/mL to about 10.0 mg/mL in a pharmaceutically acceptable liquid; or (ii) a liquid comprising platelet-derived growth factor (PDGF) at a concentration in a range of about 0.05 mg/mL to about 10.0 mg/mL in a pharmaceutically acceptable liquid. The method may further comprises the step of removing at least a portion of a previously applied matrix prior to applying a subsequent treatment. More particularly, the intramarrow bone penetration may be performed on bone that has been decorticated. The biocompatible matrix comprises, in certain embodiments, a mesh, a gauze, a sponge, a monophasic plug, a biphasic plug, a paste, a putty, a wrap, a bandage, a patch, or a pad. Furthermore, in some embodiments, the biocompatible matrix comprises a Type I collagen, a Type II collagen, a Type III collagen, bovine collagen, human collagen, porcine collagen, equine collagen, avian collagen, or combinations thereof. In some embodiments, the method further comprises applying a dressing, a skin flap, or a graft at the site after performing the initial treatment and/or a subsequent treatment.

In certain embodiments, the compositions and methods disclosed herein promote the migration and/or infiltration of at least one endogenous wound healing component to the injury site. Such components include periosteogeneic cells, angiogenic cells, stromal cells, mesenchymal cells, osteoprogenitor cells, osteoblasts, osteoclasts, platelets, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-BB, PDGF-CC, PDGF-DD, an osteogenin, and combinations thereof.

In particular embodiments, this migration and/or infiltration occurs as a result of the egress of such components from marrow into the injury site or into impaired or damaged tissue at the injury site. Accordingly, in certain embodiments, the disclosed compositions and methods promote the egress of marrow components from marrow into the injury site, and/or to damaged or impaired tissue at the injury site. Such marrow components include periosteogeneic cells, angiogenic cells, stromal cells, mesenchymal cells, osteoprogenitor cells, osteoblasts, osteoclasts, platelets, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-BB, PDGF-CC, PDGF-DD, an osteogenin, and combinations thereof. The egress of such components additionally may advantageously promote infiltration of such components into the biocompatible matrix that is applied thereto.

\Without wishing to be bound by any theory, it is believed that applying the disclosed compositions and methods facilitate, inter alia, the growth or generation of regenerative tissues at such a site. In particular, such compositions promote the growth and/or establishment of supportive tissue, which in turn supports the growth, maintenance, and ultimately the survival of a skin flap or graft that is applied to the site either concomitant with or subsequent to the application of the compositions, as described herein throughout. Furthermore, it is believed that the disclosed compositions and methods promote the growth and/or establishment of supportive tissue, inter alia, by serving as a biological and/or mechanical support that provides conditions that are suitable for proliferation, differentiation, vascularization, and angiogenesis in a particular tissue type which is to give rise to the supportive tissue. Still further, it is believed that such supportive tissue supports the skin flap or graft by, inter alia: facilitating gas, for example oxygen and/or carbon dioxide, transport and exchange; facilitating nutrient and waste product transport and exchange; facilitating laying down of granulation tissue and/or extracellular matrix components, such as glycosaminoglycans, proteoglycans, heparin sulfate, hyaluronic acid, chondroitin sulfate, karatan sulfate, any one or more of collagens I through XIII, elastin, fibronectin, laminin, nidogens, entactins, keratins, and the like, as understood in the art; facilitating expression of growth factors important for supportive tissue growth and differentiation; and/or serving as both a mechanical and a biological support for the skin flap or graft.

Moreover, in certain embodiments, the egressed marrow components, as well as regenerative tissue, supportive tissue, granulation tissue, extracellular matrix components, and the like as described above, advantageously infiltrate the biocompatible matrix that is applied to the site of the burn injury and/or applied to the area where tissue has been impaired or damaged within the site of the burn injury. This infiltration promotes the proliferation, differentiation, growth and establishment of cells and tissues that will ultimately comprise the regenerative and/or supportive tissue within the biocompatible matrix. Thus, in such embodiments the biocompatible matrix serves as a biological and/or mechanical scaffold within which regenerating and/or supportive tissue may grow and support the growth and maintenance of a skin flap or graft that is or has previously been applied to the site. During or subsequent to these processes, the biocompatible matrix is preferably resorbed and/or remodeled.

In accordance with the above, preferred biocompatible matrices as disclosed herein and/or compositions comprising such biocompatible matrices, are those which are amenable to and/or promote: absorption and/or disposition of PDGF and/or other biologically active agents as disclosed herein throughout, and/or allow for infiltration by at least one marrow component, such as periosteogeneic cells, angiogenic cells, stromal cells, mesenchymal cells, osteoprogenitor cells, osteoblasts, osteoclasts, platelets, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a fibroblast growth factor, a keratinocyte growth factor, PDGF-AB, PDGF-AA, PDGF-BB, PDGF-CC, PDGF-DD, an osteogenin, or combinations thereof. Preferred biocompatible matrices also include those that are compatible with, and promote the production of, extracellular matrix components such as those described above, and promote the establishment of granulation tissue at the site. Biocompatible matrices or compositions comprising such characteristics as described above, when applied to burn injury sites and/or to impaired, damaged and/or necrotic tissue at such sites, advantageously promote the proliferation, vascularization, and angiogenesis in regenerating and supportive tissue in accordance with the disclosed methods, and are thus preferred for use in such methods.

Methods for ascertaining the extent to which such regenerative or supportive tissue has been generated, regenerated, established, and/or formed are available in the art and may be employed in accordance with the disclosed methods. For example, methods for assessing blood flow and/or gas exchange, metabolic activity, growth factor expression, such as with various spectroscopic and/or imaging techniques, histologic and immunohistochemical assays and techniques, and the like available in the art (e.g., MRI, infrared and other spectroscopic methods, Doppler blood flow analysis, ultrasound, and the like), may be used. Similarly, oxygen saturation and/or partial pressure measurements may be made at the skin flap or graft site. Additionally, methods available in the art for assessing the extent to which either the biocompatible matrix, the injury site per se, or both, has been infiltrated by regenerative tissue may be employed. Still further, the artisan may assess the extent to which supportive tissue, such as granulation tissue, has been laid down and/or established, either within the biocompatible matrix, within the injury site per se, or both.

As used herein throughout, the term “effective amount” means the amount of the PDGF and/or another biologically active agent that may be employed in accordance with the disclosed methods that will elicit an efficacious biological or clinical response at the full thickness burn injury site, and/or in an impaired or damaged tissue therein, in a subject in need thereof.

As used herein throughout, the term “subject in need thereof” refers to a subject that has suffered a full thickness burn injury. Such a subject may be an animal, such as a bird or a mammal. Exemplary mammals include: primates, such as monkeys, apes, and human; pigs, cows, and other livestock; domesticated pets, such as dogs and cats; and other animals, such as horses. Preferably, the subject is a human.

In certain embodiments, the biocompatible matrix is resorbed and/or remodeled by infiltrating components and supportive tissues that are generated or regenerated in accordance with the disclosed methods.

In certain other embodiments, as explained below, the biocompatible matrix is not absorbed. In such embodiments, the non-resorbable matrix is optionally removed from subject after the biological and clinical benefits, such as those described herein throughout, provided by the biocompatible matrix and PDGF and/or other biologically active agents therein have been realized.

In certain other embodiments the subject develops or is at risk of developing an infection at the site of the full thickness burn injury. Moreover, in some embodiments, the subject develops or is at risk of developing at least one of the following conditions: sepsis, septic shock, necrotizing fasciitis, gangrene, or osteomyelitis. In certain embodiments, the disclosed methods further comprise disposing in the biocompatible matrix one or more subsequent effective amounts of PDGF are disposed in the biocompatible matrix. In certain embodiments, such a subsequent effective amount of PDGF is applied to the site approximately one week after performing the initial applying step. In other embodiments, the subsequent effective amount of PDGF is disposed in the biocompatible matrix at periods of time after the initial applying step of one hour, from one hour to 12 hours; from 12 hours to 24 hours; from 24 hours to seven days; from one week to two weeks; from two weeks to one month; from one month to two months; from two months to six months; from six months to one year; from one year to three years; or combinations thereof. In certain embodiments, the one or more subsequent effective amounts are periodically applied to the site. Additionally the intervals between such periodic applications of effective mounts of PDGF may be any interval ranging from approximately 12 hours to approximately 180 days, and preferably at an interval of approximately 3, 5, 7, 10, 14, 15, 21, 28, 30, 60, 90 or 180 days, or any combinations thereof. The intervals between applications may or may not be equal intervals, and may be shorter during the earlier phases of the treatment. The periodic applications of subsequent effective amounts of PDGF may be applied over a period ranging from approximately one day to approximately one year, and preferably over a period of at least approximately one week, at least approximately one month, at least approximately two months, at least approximately three months, at least approximately four months, at least approximately five months, at least approximately six months, at least approximately nine months, or at least approximately one year after initial applying step is performed.

In other embodiments, the methods further comprise applying to the site one or more subsequent compositions comprising a biocompatible matrix and an effective amount of PDGF disposed therein. The subsequent composition may be applied to the site one hour, from one hour to 12 hours, from 12 hours to 24, from 24 hours to seven days, from one week to two weeks, from two weeks to one month, from one month to two months, from two months to six months, from six months to one year, up to one year, from one year to three years or combinations thereof, after performing the initial applying step. In certain embodiments, such a subsequent composition is applied to the site approximately one week after performing the initial applying step. In certain embodiments, one or more subsequent such compositions are periodically applied to the site. Additionally the intervals between such periodic applications of such compositions may be any interval ranging from approximately 12 hours to approximately 180 days, and preferably at an interval of approximately 3, 5, 7, 10, 14, 15, 21, 28, 30, 60, 90 or 180 days, or any combinations thereof. The intervals between applications may or may not be equal intervals, and may be shorter during the earlier phases of the treatment. The periodic applications of subsequent compositions may be applied over a period ranging from approximately one day to approximately one year, and preferably over a period of at least approximately one week, at least approximately one month, at least approximately two months, at least approximately three months, at least approximately four months, at least approximately five months, at least approximately six months, at least approximately nine months, or at least approximately one year after initial applying step is performed.

In other embodiments, the method further comprises applying a dressing, a skin flap, or a graft at the site after performing the applying step. The dressing, skin flap, or graft may comprise one or more of the following: a cadaver-derived skin graft; an animal skin graft; a skin autograft; a skin allograft; a synthetic skin substitute; a dermal substitute; fibroblast-derived temporary skin substitute; an artificial skin; a reconstructive tissue matrix; an acellular tissue matrix; an acellular dermal matrix (ADM); a processed human tissue graft; a reconstructive tissue matrix; a bioengineered cell construct; a bioengineered collagen matrix; an engineered tissue graft; a cellular and collagen construct; a collagen dressing; a human bi-layered bio-engineered skin; a polyglactin mesh scaffold; a graft or composition for grafting as disclosed in, e.g., U.S. patent Ser. No. 6,979,670, the disclosure of which is hereby incorporated by reference in its entirety; a composite structure such as disclosed in, e.g., U.S. patent application Ser. No. 12/997,611 (published as US 20110091515) the disclosures of which are hereby incorporated in their entireties for all purposes; a dermal substitute; an acellular dermal matrix (ADM) as disclosed in, e.g., U.S. patent Ser. Nos. 7,972,631 and 7,358,284, the disclosures of which are hereby incorporated in their entireties for all purposes; or a polyglactin mesh scaffold. In some embodiments, an effective amount of PDGF is disposed in the dressing, the skin flap, or the graft.

Exemplary dressings or grafts that may be employed in accordance with the disclosed methods include DERMAGRAFT human fibroblast-derived dermal substitute, TRANSCYTE human fibroblast-derived temporary skin substitute, ALLODERM acellular tissue matrix, DermaMatrix® dermal matrix, Tutoplast® processed human tissue graft, Strattice™ reconstructive tissue matrix, PURAPLY collagen dressing, INTEGRA Artificial Skin, DeNovo NT graft marketed by ZIMMER, DeNovo ET Engineered Tissue Graft marketed by Zimmer, BIOBRANE temporary composite wound dressing, APLIGRAF bioengineered cell construct, CELTX living cellular and collagen construct, FORTAFLEX bioengineered collagen matrix, FORTAGEN collagen construct, or VC101 human bi-layered bio-engineered skin.

Turning now to components that can be included in various embodiments of the present invention, which include compositions comprising a biocompatible matrix and an effective amount of PDGF.

### PDGF

In one aspect, a composition for use in the disclosed methods comprises a biocompatible matrix and platelet derived growth factor (PDGF) disposed therein. In some embodiments, the PDGF is present at a concentration ranging from about 0.01 mg/ml to about 10 mg/ml, from about 0.05 mg/ml to about 5 mg/ml, from about 0.1 mg/ml to about 1.0 mg/ml. PDGF may be present at any concentration within these stated ranges. In other embodiments, PDGF is present at any one of the following concentrations or within any one of the following concentration ranges: at a concentration from about 0.01 mg/ml to about 10 mg/ml; from about 0.05 mg/ml to about 5 mg/ml; from 0.1 to about 1.0 mg/ml; from about 0.2 to about 0.75 mg/ml; from about 0.25 to about 0.6 mg/ml; from about 0.01 to about 0.5 mg/ml; from about 0.01 mg/ml to about 0.4 mg/ml; from about 0.01 mg/ml to about 0.3 mg/ml; from about 0.01 mg/ml to about 0.3 mg/ml; from about 0.01 mg/ml to about 0.2 mg/ml; from about 0.05 mg/ml to about 0.3 mg/ml; about 0.01 mg/ml; about 0.02 mg/ml; about 0.03 mg/ml; about 0.04 mg/ml; about 0.05 mg/ml; about 0.06 mg/ml; about 0.07 mg/ml; about 0.08 mg/ml; about 0.09 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.0 mg/ml; 0.1 mg/ml; 0.3 mg/ml; 1.0 mg/ml; and combinations thereof.

It is to be understood that these concentrations are simply examples of particular embodiments, and that the concentration of PDGF may be within any of the concentration ranges stated above.

Various amounts of PDGF may be used in the compositions of the present invention. Amounts of PDGF that could be used include amounts in the following ranges: about 1 μg to about 50 mg, about 10 μg to about 25 mg, about 100 μg to about 10 mg, and about 250 μg to about 5 mg.

The concentration of PDGF or other growth factors in embodiments of the present invention can be determined by using an enzyme-linked immunoassay as described in U.S. Pat. Nos. 6,221,625, 5,747,273, and 5,290,708, or any other assay known in the art for determining PDGF concentration. When provided herein, the molar concentration of PDGF is determined based on the molecular weight of PDGF dimer (e.g., PDGF-BB; MW about 25 kDa).

In embodiments of the present invention, PDGF comprises PDGF homodimers and heterodimers, including PDGF-AA, PDGF-BB, PDGF-AB, PDGF-CC, PDGF-DD, and mixtures and derivatives thereof. In one embodiment, PDGF comprises PDGF-BB. In another embodiment PDGF comprises a recombinant human PDGF, such as rhPDGF-BB.

PDGF, in some embodiments, can be obtained from natural sources. In other embodiments, PDGF can be produced by recombinant DNA techniques. In other embodiments, PDGF or fragments thereof may be produced using peptide synthesis techniques known to one of skill in the art, such as solid phase peptide synthesis. When obtained from natural sources, PDGF can be derived from biological fluids. Biological fluids, according to some embodiments, can comprise any treated or untreated fluid associated with living organisms including blood.

Biological fluids, in another embodiment, can also comprise blood components including platelet concentrate (PC), apheresed platelets, platelet-rich plasma (PRP), platelet-poor plasma (PPP), platelet-free plasma, plasma, serum, fresh frozen plasma (FFP), bone marrow, and buffy coat (BC). Biological fluids, in a further embodiment, can comprise platelets separated from plasma and resuspended in a physiological fluid.

When produced by recombinant DNA techniques, a DNA sequence encoding a single monomer (e.g., PDGF B-chain or A-chain), in some embodiments, can be inserted into cultured prokaryotic or eukaryotic cells for expression to subsequently produce the homodimer (e.g. PDGF-BB or PDGF-AA). In other embodiments, a PDGF heterodimer can be generated by inserting DNA sequences encoding for both monomeric units of the heterodimer into cultured prokaryotic or eukaryotic cells and allowing the translated monomeric units to be processed by the cells to produce the heterodimer (e.g. PDGF-AB). Commercially available cGMP recombinant PDGF-BB can be obtained commercially from Chiron Corporation (Emeryville, Calif.). Research grade rhPDGF-BB can be obtained from multiple sources including R&D Systems, Inc. (Minneapolis, Minn.), BD Biosciences (San Jose, Calif.), and Chemicon, International (Temecula, Calif.).

In embodiments of the present invention, PDGF comprises PDGF fragments. In one embodiment rhPDGF-B comprises the following fragments: amino acid sequences 1-31, 1-32, 33-108, 33-109, and/or 1-108 of the entire B chain. The complete amino acid sequence (1-109) of the B chain of PDGF is provided in FIG. 15 of U.S. Pat. No. 5,516,896. It is to be understood that the rhPDGF compositions of the present invention may comprise a combination of intact rhPDGF-B (1-109) and fragments thereof. Other fragments of PDGF may be employed such as those disclosed in U.S. Pat. No. 5,516,896. In accordance with one embodiment, the rhPDGF-BB comprises at least 65% of intact rhPDGF-B (1-109). In accordance with other embodiments, the rhPDGF-BB comprises at least 75%, 80%, 85%, 90%, 95%, or 99% of intact rhPDGF-B (1-109).

In some embodiments of the present invention, PDGF can be purified. Purified PDGF, as used herein, comprises compositions having greater than about 95% by weight PDGF prior to incorporation in solutions of the present invention. The solution may be any pharmaceutically acceptable solution. In other embodiments, the PDGF can be substantially purified. Substantially purified PDGF, as used herein, comprises compositions having about 5% to about 95% by weight-PDGF prior to incorporation into solutions of the present invention. In one embodiment, substantially purified PDGF comprises compositions having about 65% to about 95% by weight PDGF prior to incorporation into solutions of the present invention. In other embodiments, substantially purified PDGF comprises compositions having about 70% to about 95%, about 75% to about 95%, about 80% to about 95%, about 85% to about 95%, or about 90% to about 95%, by weight PDGF, prior to incorporation into solutions of the present invention. Purified PDGF and substantially purified PDGF may be incorporated into biocompatible matrices.

In a further embodiment, PDGF can be partially purified. Partially purified PDGF, as used herein, comprises compositions having PDGF in the context of PRP, FFP, or any other blood product that requires collection and separation to produce PDGF. Embodiments of the present invention contemplate that any of the PDGF isoforms provided herein, including homodimers and heterodimers, can be purified or partially purified. Compositions of the present invention containing PDGF mixtures may contain PDGF isoforms or PDGF fragments in partially purified proportions. Partially purified and purified PDGF, in some embodiments, can be prepared as described in U.S. patent application Ser. No. 11/159,533 (Publication No: 20060084602).

In some embodiments, PDGF is provided in a pharmaceutically acceptable liquid, such as a buffer solution. Buffers suitable for use in PDGF buffer solutions of the present invention can comprise, but are not limited to, carbonates, phosphates (e.g. phosphate buffered saline), histidine, acetates (e.g. sodium acetate), acidic buffers such as acetic acid and HCl, and organic buffers such as lysine, Tris buffers (e.g. tris(hydroxymethyl)aminoethane), N-2-hydroxyethylpiperazine-N′-2-ethanesulfonic acid (HEPES), and 3-(N-morpholino) propanesulfonic acid (MOPS). Buffers can be selected based on biocompatibility with PDGF and the buffer's ability to impede undesirable protein modification. Buffers can additionally be selected based on compatibility with host tissues. In one embodiment, sodium acetate buffer is used. The buffers may be employed at different molarities, for example about 0.1 mM to about 100 mM, about 1 mM to about 50 mM, about 5 mM to about 40 mM, about 10 mM to about 30 mM, or about 15 mM to about 25 mM, or any molarity within these ranges. In some embodiments, an acetate buffer is employed at a molarity of about 20 mM.

In another embodiment, solutions comprising PDGF are formed by solubilizing lyophilized PDGF in water, wherein prior to solubilization the PDGF is lyophilized from an appropriate buffer.

Solutions comprising PDGF, according to embodiments of the present invention, can have a pH ranging from about 3.0 to about 8.0. In one embodiment, a solution comprising PDGF has a pH ranging from about 5.0 to about 8.0, more preferably about 5.5 to about 7.0, most preferably about 5.5 to about 6.5, or any value within these ranges. The pH of solutions comprising PDGF, in some embodiments, can be compatible with the prolonged stability and efficacy of PDGF or any other desired biologically active agent. PDGF is generally more stable in an acidic environment. Therefore, in accordance with one embodiment the present invention comprises an acidic storage formulation of a PDGF solution. In accordance with this embodiment, the PDGF solution preferably has a pH from about 3.0 to about 7.0, and more preferably from about 4.0 to about 6.5. The biological activity of PDGF, however, can be optimized in a solution having a neutral pH range. Therefore, in a further embodiment, the present invention comprises a neutral pH formulation of a PDGF solution. In accordance with this embodiment, the PDGF solution preferably has a pH from about 5.0 to about 8.0, more preferably about 5.5 to about 7.0, most preferably about 5.5 to about 6.5. In accordance with a method of the present invention, an acidic PDGF solution is reformulated to a neutral pH composition, wherein such composition is then used to promote healing, regeneration, vascularization and angiogenesis in fascia, muscle, tendon, periosteum, and/or bone in accordance with the disclosed methods. In accordance with a preferred embodiment of the present invention, the PDGF utilized in the solutions is rhPDGF-BB.

In some embodiments, the pH of the PDGF containing solution may be altered to optimize the binding kinetics of PDGF to a matrix substrate or linker If desired, as the pH of the material equilibrates to adjacent material, the bound PDGF may become labile.

The pH of solutions comprising PDGF, in some embodiments, can be controlled by the buffers recited herein. Various proteins demonstrate different pH ranges in which they are stable. Protein stabilities are primarily reflected by isoelectric points and charges on the proteins. The pH range can affect the conformational structure of a protein and the susceptibility of a protein to proteolytic degradation, hydrolysis, oxidation, and other processes that can result in modification to the structure and/or biological activity of the protein.

In some embodiments, solutions comprising PDGF can further comprise additional components, such as other biologically active agents. In other embodiments, solutions comprising PDGF can further comprise cell culture media, other stabilizing proteins such as albumin, antimicrobial agents, such as antibiotics, antiseptics, antibacterial agents, and the like, protease inhibitors [e.g., ethylenediaminetetraacetic acid (EDTA), ethylene glycol-bis(beta-aminoethylether)-N,N,N′,N′-tetraacetic acid (EGTA), aprotinin, ε-aminocaproic acid (EACA), etc.] and/or other growth factors such as fibroblast growth factors (FGFs), transforming growth factors (e.g., transforming growth factor-β1 (TGF-β1), transforming growth factor-β2 (TGF-β2), or transforming growth factor-α (TGF-α)), epidermal growth factors (EGFs), transforming growth factors (TGFs), keratinocyte growth factors (KGFs), insulin-like growth factors (IGFs) (e.g., insulin-like growth factor I (IGF-I) and an insulin-like growth factor II (IGF-II)), bone morphogenetic proteins (BMPs), osteogenins, or other PDGFs including compositions of PDGF-AA, PDGF-BB, PDGF-AB, PDGF-CC and/or PDGF-DD. Exemplary antibiotics for use in accordance with the disclosed methods include penicillin, ampicillin, penicillin G, clindamycin (Cleocin), Ceftriaxone (Rocephin), erythromycin, gentamicin (Garamycin), clindamycin (Cleocin), metronidazole (Flagyl), Ampicillin-sulbactam (Unasyn), ticarcillin-clavulanate potassium (Timentin), piperacillin-tazobactam (Zosyn), nafcillin (Unipen), Imipenem-cilastatin (Primaxin), Doripenem, a β-lactam, a β-lactamase inhibitor, antipseudomonal cephalosporin, ceftazidime (Fortaz), clindamycin, metronidazole, Vancomycin (Vancocin) an aminoglycoside, aztreonam (Azactam), amphotericin B (Abelcet), a third-generation cephalosporin, and combinations thereof.

In addition to solutions comprising PDGF, compositions of the present invention also comprise a biocompatible matrix into which PDGF solutions may be disposed.

### Biocompatible Matrix

A biocompatible matrix, according to embodiments of the present invention provides, inter alia, a framework or scaffold for new tissue growth to occur at a site of a full thickness burn injury, including integumental tissues, subcutaneous tissue, fascia, vascular tissue, muscle tissue, nerve tissue, vascular tissue, tendon, periosteal tissue, and/or bone tissue. A biomechanical matrix in accordance with the present invention also, inter alia, promotes the healing and regeneration of such tissues that have been impaired or damaged as a result of such burn injuries. A biomechanical matrix in accordance with the present invention also promotes vasularization, angiogenesis, in such regenerating tissues, and promotes growth of a vascularized tissue bed and/or supportive tissue that supports the survival of a skin flap or graft that is applied at the injury site.

Biocompatible matrices suitable for use in accordance with the disclosed methods may comprise a synthetic component, a natural component, or combinations thereof. Such components may comprise proteins, polysaccharides, nucleic acids, carboyhydrates, or synthetic polymers, or mixtures thereof. Non-limiting examples of suitable components from which biocompatible matrices may be prepared in accordance with the present invention include: elastins, polysaccharides, nucleic acids, carbohydrates, proteins, polyurethanes, siloxanes, polysiloxanes, collagens, glycosaminoglycans, oxidized regenerated cellulose (ORC), ethylene diamine tetraacetic acid (EDTA),a poly(lactic-co-glycolitic acid (PLGA), carboxymethylcellulose, granulated collagen-glycosaminoglycan composites, methylcelluloses, hydroxypropyl methylcellulose, hydroxyethyl cellulose alginic acid, poly(a-hydroxy acids), poly(lactones), poly(amino acids), poly(anhydrides), poly(orthoesters), poly(anhydride-co-imides), poly(orthocarbonates), poly(a-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), polyvinyl alcohol), poly(vinylpyrrolidone), poly(ethyloxazoline), poly(ethylene oxide)-co-poly(propylene oxide) block copolymers, poly(ethylene terephthalate)polyamidearabic gums, guar gums, xantham gums, gelatins, chitin, chitosan, chitosan acetate, chitosan lactate, chondroitin sulfate, N,O-carboxymethyl chitosan, dextrans, fibrin glue, glycerol, hyaluronic acid, sodium hyaluronate, celluloses, glucosamines, proteoglycans, starches, lactic acid, pluronics, sodium glycerophosphate, glycogens, keratins, and silks.

Collagens suitable for use in preparing a biocompatible matrix for use accordance with the disclosed methods include a Type I collagen, a Type II collagen, or a Type III collagen a Type IV collagen, a Type V collagen, a Type VI collagen, a Type V collagen, a Type VI collagen, a Type VII collagen, a Type VIII collagen, or combinations thereof. Furthermore, the collagen may be derived from any of a variety of mammalian species; thus suitable collagens are, for example, bovine collagen, human collagen, porcine collagen, equine collagen, and avian collagen. Preferably, the biocompatible matrix comprises a Type I collagen, a Type II collagen, or a Type III collagen. In certain embodiments, the biocompatible matrix comprises a bovine Type I collagen.

A biocompatible matrix, in some embodiments, comprises at least one calcium phosphate. In other embodiments, a biocompatible matrix can comprise a plurality of calcium phosphates. Calcium phosphates suitable for use as a biocompatible matrix, in embodiments of the present invention, have a calcium to phosphorus atomic ratio ranging from 0.5 to 2.0. In some embodiments the biocompatible matrix comprises an allograft such as DFDBA or particulate DBM.

Non-limiting examples of calcium phosphates suitable for use as biocompatible matrices comprise amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate, β-TCP, hydroxyapatite (OHAp), poorly crystalline hydroxyapatite, tetracalcium phosphate (TTCP), heptacalcium decaphosphate, calcium metaphosphate, calcium pyrophosphate dihydrate, carbonated calcium phosphate, and calcium pyrophosphate.

A biocompatible matrix, in some embodiments, comprises β-TCP. β-TCP, according to some embodiments, can comprise a porous structure having multidirectional and interconnected pores of varying diameters. In some embodiments, β-TCP comprises a plurality of pockets and non-interconnected pores of various diameters in addition to the interconnected pores. The porous structure of β-TCP, in one embodiment, comprises macropores having diameters ranging from about 100 μm to about 1 mm, mesopores having diameters ranging from about 10 μm to about 100 μm, and micropores having diameters less than about 10 μm. Macropores and micropores of the β-TCP can facilitate tissue in-growth including osteoinduction and osteoconduction while macropores, mesopores and micropores can permit fluid communication and nutrient transport to support tissue and bone regrowth throughout the β-TCP biocompatible matrix. β-TCP, in some embodiments, can have a porosity greater than 25%. In other embodiments, β-TCP can have a porosity greater than 50%. In a further embodiment, β-TCP can have a porosity greater than 90%.

In some embodiments, a biocompatible matrix comprises β-TCP particles. β-TCP particles, in one embodiment, have an average diameter ranging from about 1 μm to about 5 mm. In other embodiments, β-TCP particles have an average diameter ranging from about 250 μm to about 750 nm. In another embodiment, β-TCP particles have an average diameter ranging from about 100 μm to about 400 nm. In a further embodiment, β-TCP particles have an average diameter ranging from about 75 μm to about 300 nm. In additional embodiments, β-TCP particles have an average diameter less than 25 nm and, in some cases, sizes less than 1 mm.

A biocompatible matrix comprising a β-TCP, in some embodiments, is provided in a shape suitable for application to a burn injury site (e.g., a sphere, a cylinder, or a block). In other embodiments, a β-TCP biocompatible matrix is moldable, extrudable, and/or flowable thereby facilitating application of the matrix at or in tissue that has been damaged or impaired as a burn injury site in accordance with the disclosed methods. Flowable matrices may be applied through syringes, tubes, or spatulas.

A β-TCP biocompatible matrix, according to some embodiments, is bioresorbable. In one embodiment, a β-TCP biocompatible matrix can be at least 75% resorbed one year subsequent to in vivo implantation. In another embodiment, a β-TCP biocompatible matrix can be greater than 90% resorbed one year subsequent to in vivo implantation.

A biocompatible matrix suitable for use in accordance with the disclosed methods may comprise, or may further comprise, in vitro-prepared tissue or in vitro-prepared dermal tissue, or biological components made thereby. An in vitro-prepared tissue may be synthesized by any of a variety of cells, including cells derived from skin tissue, such as fibroblasts, or cells derived from other tissues (e.g., tendon, ligament, synovium, muscle, mucosa, bone marrow) that are capable of producing natural products that are found in wound sites and that contribute to wound healing and/or tissue repair. In some cases these cells may be fibroblasts or other cell types such as endothelial cells or stem cells that are capable of either directly producing factors involved in wound healing, or are capable of differentiating into a cell type, or being induced to differentiate into a cell, that is capable of producing products that are involved in wound healing. Cell lines and genetically-modified cells may also be used provided that they are capable of producing components involved in wound healing and or tissue regeneration. Examples of cells that may be utilized to produce an in vitro-prepared tissue, include, but are not limited to, fibroblasts, stromal cells (e.g., marrow-derived stromal cells), and mesenchymal stem cells. When stromal cells or mesenchymal stem cells are used, it may be necessary, or desirable, to first induce the cells to differentiate or to separate out a subpopulation of cells that exhibit a fibroblastic phenotype. Subpopulations of cells may be separated by any of a variety of methods known in the art, including, for example, by FACS, by eliminating cells that do not attach to a substrate over a predetermined period of time, or by selecting cells that bind to a certain epitope which is characteristic of skin tissue (e.g., an RGD-peptide). In some embodiments, the cells of the in vitro-prepared tissue comprise fibroblasts. In some embodiments, the cells of the in vitro-prepared tissue consist only of fibroblasts. In some embodiments, the cells of the in vitro-prepared tissue do not comprise keratinocytes, endothelial cells, or Langerhans cells. In some embodiments, the in vitro-prepared tissue comprises synovium, cartilage, bone, tendon, ligament, or muscle producing cells.

In some embodiments, the in vitro-prepared tissue does not comprise living cells. For example, the in vitro-prepared tissue may be subjected to storage conditions that are not suitable for the maintenance of viable cells, as is done for the product TransCyte, which is a Human Fibroblast Derived Temporary Skin Substitute.

In vitro-prepared tissue typically comprises a variety of different extracellular matrix proteins, including those that are commonly found in healing wounds and/or skin. Non-limiting examples of such proteins include collagen, including any one of collagen Type Ito XIII, elastin, laminin, fibronectin, and tenascin. In vitro-prepared tissue also typically comprises one or more glycosaminoglycans that are commonly found in normal tissues and in healing wounds or in sites of active tissue repair and remodeling. Non-limiting examples of such glycosaminoglycans are selected from: chondroitin sulfate, dermatan sulfate, keratan sulfate, heparin, heparan sulfate, hyaluronan, versican, decorin, betaglycan, and syndecan. Certain growth factors and cytokines are also often present in in vitro-prepared tissue. Examples of such growth factors and cytokines include, for example, IL-8, IL-6, IL-1.alpha., PDGF-A, IGF, KGF, HBEGF, TGF-.alpha., TGF-.beta.1, TGF-.beta.3, VEGF, G-CSF, Angiopoietin I, acidic FGF, basic FGF and HGF. In particular, in vitro-prepared tissue often includes components (e.g., growth factors) that stimulate angiogenesis. For example, in vitro-prepared tissue typically comprises diffusible and/or extracellular matrix-binding forms of VEGF, or basic FGF. While it is to be appreciated that matrix proteins, glycosaminoglycans, growth factors, and other compounds present in the tissue are typically expressed from the cells that are used to produce the tissue in vitro, exogenous sources may also be used. For example, soluble growth factors, such as, for example, TGF-.beta.1, TGF-.beta.3, VEGF, PDGF (PDGF-AA, -BB, -AB, -CC, -DD) may be exogenously added to the tissue (e.g., as recombinant proteins or proteins isolated from natural sources) and remain resident in the tissue. Extracellular matrix proteins may also be supplied exogenously. For example, collagen may be supplied as a matrix component on which the cells which produce the tissue are seeded.

In some embodiments, in vitro-prepared dermal tissues are provided for use in accordance with the disclosed methods. The term “in vitro-prepared dermal tissue”, as used herein, refers to a tissue that resembles in whole, or in part, skin, or a layer of skin (e.g. dermis) that has been synthesized in a controlled environment outside of a living organism. In some embodiments, the in vitro-prepared dermal tissue resembles in whole, or in part, only the dermis of skin. In some embodiments, the in vitro-prepared tissue is a tissue (e.g., a fibrotic tissue) that resembles a component of skin below the epidermal layer.

DERMAGRAFT (Interactive Wound Dressing) is an example of a commercially available in vitro-prepared dermal tissue and may be used in the methods disclosed herein (Gail K. Naughton, Dermal Equivalents, Chapter 63, Principles of Tissue Engineering, Second Edition, Academic Press, Copyright 2000; and Jonathan N. Mansbridge, Growth factors secreted by fibroblasts: role in healing diabetic foot ulcers, Diabetes, Obesity and Metabolism, 1, 1999, 265-279, the contents of which relating to DERMAGRAFT are incorporated herein by reference). DERMAGRAFT is a cryopreserved human fibroblast-derived dermal substitute that is composed of fibroblasts, extracellular matrix, and a bioabsorbable scaffold. DERMAGRAFT is manufactured from human fibroblast cells derived from newborn foreskin tissue. During the manufacturing process, the human fibroblasts are seeded onto a bioabsorbable polyglactin mesh scaffold. The fibroblasts proliferate to fill the interstices of this scaffold and secrete various factors, including collagen, matrix proteins, growth factors, and cytokines. This creates a three-dimensional human dermal substitute that contains metabolically active, living cells and that is rich in human matrix proteins including Type I collagen (typically 80% total protein by weight) and various proteoglycans. DERMAGRAFT does not contain macrophages, lymphocytes, blood vessels, or hair follicles. The fibroblasts that exist in DERMAGRAFT remain viable (i.e., alive) after thawing. The human fibroblast cells are from a qualified cell bank, which has been extensively tested for animal viruses, retroviruses, cell morphology, karyology, isoenzymes, and tumorigenicity. Reagents used in the manufacture of DERMAGRAFT are tested and found free from viruses, retroviruses, endotoxins, and mycoplasma before use. DERMAGRAFT is manufactured with sterile components under aseptic conditions within the final package. Prior to release for use, each lot of DERMAGRAFT is evaluated by USP Sterility (14-day), endotoxin, and mycoplasma tests. DERMAGRAFT is supplied frozen in a clear bag containing one piece of approximately 2 in by 3 in (5 cm by 7.5 cm) for a single-use application. The product is stored at −75.degree. C.+−.10.degree. C. (−103.degree. F.+−.8.degree. F.) and is delivered to customers in shipping containers packed with dry ice. DERMAGRAFT has been approved as a Class III medical device by the Food and Drug Administration (FDA PMA P000036) as a therapy for the treatment of full-thickness non-healing diabetic foot ulcers and is manufactured and marketed by Advanced BioHealing Inc. In addition, DERMAGRAFT is currently under investigation as a Class III medical device for the treatment of Venus Leg Ulcers (VLU) under IDE G090056.

Other in vitro-prepared tissues, in vitro-prepared dermal tissues, biological components made thereby or derived therefrom, as well as devices and methods for preparing, using, and implanting such tissues in accordance with the disclosed methods are further disclosed in U.S. patent application Ser. No. 13/042,295, published as U.S. patent application number U.S. 2011/0245929, the disclosures of which are hereby incorporated by references in their entireties for all purposes.

The biocompabile matrix may be in the form of a mesh, a gauze, a sponge, a monophasic plug, a biphasic plug, a paste, a putty, a wrap, a bandage, a patch, or a pad. Fibrous collagen suitable for use in collagen patches or pads demonstrate sufficient mechanical properties, including wet tensile strength, to withstand suturing and hold a suture without tearing. In one embodiment, a collagen patch or pad has a density ranging from about 0.75 g/cm³ to about 1.5 g/cm³. Additionally, a collagen patch or pad for use in some embodiments of the present invention is porous and operable to absorb water in an amount ranging from about lx to about 15× the mass of the collagen patch.

In some embodiments, a biocompatible matrix comprises porous structure. Porous biocompatible matrices, according to some embodiments, can comprise pores having diameters ranging from about 1 μm to about 1 mm. In one embodiment, a biocompatible matrix comprises macropores having diameters ranging from about 100 μm to about 1 mm. In another embodiment, a biocompatible matrix comprises mesopores having diameters ranging from about 10 μm to about 100 μm. In a further embodiment, a biocompatible matrix comprises micropores having diameters less than about 10 μm. Embodiments of the present invention contemplate biocompatible matrixs comprising macropores, mesopores, and micropores or any combination thereof.

A porous biocomatible matrix, in one embodiment, has a porosity greater than about 25%. In another embodiment, a porous biocomatible matrix has a porosity greater than about 50%. In a further embodiment, a porous biocompatible matrix has a porosity greater than about 90%.

In some embodiments, a biocompatible matrix comprises a plurality of particles. A biocompatible matrix, for example, can comprise a plurality of calcium phosphate particles. Biocompatible matrices, in one embodiment, have an average diameter ranging from about 1 μm to about 5 mm. In other embodiments, particles have an average diameter ranging from about 250 μm to about 750 μm. Biocompatible matrices, in another embodiment, can have average diameter ranging from about 100 μm to about 400 μm. In a further embodiment, the particles have an average diameter ranging from about 75 pm to about 300 μm. In additional embodiments, biocompatible matrices have an average diameter less than about 1 μm and, in some cases, less than about 1 mm.

Biocompatible matrices, according to some embodiments, can be provided in a shape suitable for application to a burn injury site (e.g., a sphere, a cylinder, or a block). In other embodiments, biocompatible matrices are moldable, extrudable, and/or injectable. Moldable biocompatible matrices can facilitate efficient placement of compositions of the present invention in and around various tissues that have been damaged or impaired at burn injury sites, including fascia, muscle, tendon, periosteum, and bone. In some embodiments, moldable biocompatible matrices are applied to such tissues with a spatula or equivalent device. In some embodiments, biocompatible matrices are flowable. Flowable biocompatible matrices, in some embodiments, can be applied to a site of a full thickness burn injury, and/or to damaged or impaired tissues at such a site, through a syringe and needle or cannula.

In some embodiments, biocompatible matrices are bioresorbable. In accordance with the disclosed methods, “bioresorbable” refers to the ability of the biocompatible matrix to be resorbed or remodeled in vivo. The resorption process involves degradation and elimination of the original biocompatible matrix through the action of body fluids, proteases or other enzymes enzymes, or cells. The resorbed materials may be used by the host in the formation of new tissue, or it may be otherwise re-utilized by the host, or it may be excreted. Thus, a biocompatible matrix, in one embodiment, can be resorbed within one year of in vivo implantation. In another embodiment, a biocompatible matrix can be resorbed within 1, 3, 6, or 9 months of in vivo implantation. Bioresorbability is dependent on: (1) the nature of the matrix material (i.e., its chemical make up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors. However, in some embodiments, the biocompatible matrix is not resorbable.

Whether the biocompatible matrix is resorbable or not resorbable, the biocompatible matrix may serve as a depot or delivery device into which PDGF and/or other biologically active agents may be disposed. The disposed PDGF and/or other biologically active agents may then advantageously exit the matrix over time, providing for sustained release of such disposed agents over a protracted time course. Accordingly, sustained release of such agents may persist from about six hours to about one year. Additionally, sustained delivery of such agents may persist from about six hours to about one day, from about one day to about three days, from about one day to about one week, from about one week to about two weeks, from about one week to about one month, from about one month to about two months, from about one month to about three months, from about three months to about six months, from about three months to about nine months, from about three months to about one year, and combinations thereof. Further, intervals of sustained release time frames include approximately 1, 3, 5, 7, 10, 14, 15, 21, 28, 30, 60, 90 or 180 days, or any combinations thereof.

In some embodiments, a biocompatible matrix comprises a collagen-comprising mesh, gauze, sponge, monophasic plug, biphasic plug, paste, putty, wrap, bandage, patch, or pad. Such biocompatible matrices, in one embodiment of the present invention, comprises a fibrous collagen such as soluble type I bovine collagen or a type I human collagen. In another embodiment, a fibrous collagen comprises type II or type III collagen. Fibrous collagen suitable for use in such formats demonstrate sufficient mechanical properties, including wet tensile strength ranging from about 0.75 pounds to about 5 pounds. In one embodiment, a collagen patch or pad has a density ranging from about 0.75 g/cm³ to about 1.5 g/cm³. Additionally, a collagen patch or pad for use in some embodiments of the present invention is porous and operable to absorb water in an amount ranging from about 1× to about 15× the mass of the collagen patch.

Biocompatible matrices, according to some embodiments, can comprise materials operable to promote cohesion between combined substances. Additionally, such materials, for example, can promote adhesion between particles of a biocompatible matrix in the formation of a biocompatible matrix. In certain embodiments, such materials provide a framework for new tissue growth to occur, in accordance with the disclosed methods.

In some embodiments, a biocompatible matrix, or a component thereof as described herein throughout, is water-soluble. In certain embodiments, a water-soluble component of a biocompatible matrix can dissolve from the remainder of the biocompatible matrix shortly after its implantation, thereby introducing macroporosity into the biocompatible matrix. Macroporosity, as discussed herein, can promote infiltration of inter alia, stromal cells, mesenchymal cells and other cells and blood or marrow components that facilitate vascularization and/or angiogenesis or regenerating tissue, and/or promote healing and regeneration of damaged or impaired tissues at a burn injury site.

A biocompatible matrix can be flowable, moldable, and/or extrudable. Such biocompatible matrices can be molded into the desired implant shape or can be molded to the contours of the injury site or of a particular impaired or damaged tissue in the site. In one embodiment, a biocompatible matrix in paste or putty form can be injected into such a site or tissue with a syringe or cannula.

In some embodiments, a biocompatible matrix does not harden and retains a flowable and moldable form subsequent to application to the injury site or of a particular impaired or damaged tissue in the site. In other embodiments, a biocompatible matrix can harden subsequent to application to the injury site or of a particular impaired or damaged tissue in the site, thereby reducing matrix flowability and moldability.

A biocompatible matrix, in some embodiments, can also be provided in a predetermined shape including a block, sphere, or cylinder or any desired shape, for example a shape defined by a mold or a site of application.

In some embodiments, a biocompatible matrix can comprise a β-TCP and a collagen. β-TCP materials suitable for combination with a collagen are consistent with those provided hereinabove. The collagen component of such a biocompatible matrix, in some embodiments, comprises any type of collagen, including Type I, Type II, and Type III collagens. In one embodiment, a collagen comprises a mixture of collagens, such as a mixture of Type I and Type II collagen. In other embodiments, a collagen is soluble under physiological conditions. Other types of collagen present in bone or musculoskeletal tissues may be employed. Recombinant, synthetic and naturally occurring forms of collagen may be used in the present invention.

A biocompatible matrix, according to some embodiments, comprises a plurality of β-TCP particles adhered to one another with a collagen. In one embodiment, β-TCP particles suitable for combination with a collagen have an average diameter ranging from about 1 μm to about 5 mm. In another embodiment, β-TCP particles suitable for combination with a collagen have an average diameter ranging from about 1 μm to about 1 mm. In other embodiments, β-TCP particles have an average diameter ranging from about 200 μm to about 3 mm or about 200 μm to about 1 mm, or about 1 mm to about 2 mm. In some embodiments, β-TCP particles have an average diameter ranging from about 250 μm to about 750 μm. β-TCP particles, in other embodiments, have an average diameter ranging from about 100 μm to about 400 μm. In a further embodiment, β-TCP particles have an average diameter ranging from about 75 μm to about 300 μm. In additional embodiments, β-TCP particles have an average diameter less than about 25 μm and, in some cases, less than about 1 mm.

β-TCP particles, in some embodiments, can be adhered to one another by the collagen so as to produce a biocompatible matrix having a porous structure. In some embodiments, a biocompatible matrix comprising β-TCP particles and a collagen can comprise pores having diameters ranging from about 1 μm to about 1 mm. A biocompatible matrix comprising β-TCP particles and a collagen can comprise macropores having diameters ranging from about 100 μm to about 1 mm, mesopores having diameters ranging from about 10 μm to 100 μm, and micropores having diameters less than about 10 μm.

A biocompatible matrix comprising β-TCP particles and a collagen can have a porosity greater than about 25%. In another embodiment, the biocompatible matrix can have a porosity greater than about 50%. In a further embodiment, the biocompatible matrix can have a porosity greater than about 90%.

A biocompatible matrix comprising β-TCP particles and a collagen, in some embodiments, can comprise a collagen in an amount ranging from about 5 weight percent to about 50 weight percent of the matrix. In other embodiments, a collagen can be present in an amount ranging from about 10 weight percent to about 40 weight percent of the biocompatible matrix. In another embodiment, a collagen can be present in an amount ranging from about 15 weight percent to about 35 weight percent of the biocompatible matrix. In a further embodiment, a collagen can be present in an amount of about 20 weight percent of the biocompatible matrix.

A biocompatible matrix comprising β-TCP particles and a collagen, according to some embodiments, can be flowable, moldable, and/or extrudable. In such embodiments, the biocompatible matrix can be in the form of a paste or putty. A paste or putty can be molded into the desired implant shape or can be molded to the contours of the injury site or of a particular impaired or damaged tissue at the injury site. In one embodiment, a biocompatible matrix in paste or putty form comprising β-TCP particles and a collagen can be injected into an injury site or onto a particular impaired or damaged tissue at the injury site with a syringe or cannula.

In some embodiments, a biocompatible matrix in paste or putty form comprising β-TCP particles and a collagen can retain a flowable and moldable form when applied to an injury site. In other embodiments, the paste or putty can harden subsequent to application, thereby reducing matrix flowability and moldability.

A biocompatible matrix comprising β-TCP particles and a collagen, in some embodiments, can be provided in a predetermined shape such as a block, sphere, or cylinder.

A biocompatible matrix comprising β-TCP particles and a collagen can be resorbable. In one embodiment, a biocompatible matrix comprising β-TCP particles and a collagen can be at least 75% resorbed one year subsequent to in vivo implantation. In another embodiment, a biocompatible matrix comprising β-TCP particles and a collagen can be greater than 90% resorbed one year subsequent to in vivo implantation.

Other exemplary such biocompatible matrices which may be employed in accordance with the disclosed methods include AUGMENT Rotator Cuff Graft or another biocompatible matrix as disclosed in, for example U.S. patent application Ser. No. 11/772,646, published as US 2008/0027470, the disclosures of which are hereby incorporated by reference in their entirety for all purposes; COLLATAPE; COLLACOTE; SOLOSITE; an INTEGRA Dermal Regeneration Template; an INTEGRA Meshed Bilayer Wound Matrix; a HELISTAT Absorbable Collagen Hemostatic Sponge; a HELITENE Absorbable Collagen Hemostatic Agent; an INTEGRA Flowable Wound Matrix; Matrix Collagen Particles™ Wound Dressing; Matrix Collagen Sponge™ Wound Dressing; OssiMend™ Bone Graft Matrix mineral:collagen composite; OssiMend™ Block Bone Graft Matrix mineral:collagen composite; OssiMend™ Putty Bone Graft Matrix mineral:collagen composite; OssiPatch™ Collagen Bone Healing Protective Sheet; NeuroMatrix™ Collagen Nerve Conduit; Neuroflex™ Flexible Collagen Nerve Conduit; NeuroMend™ Collagen Wrap Conduit; Collatene™ Fibrillar Collagen Dental Dressing; SynOss™ Synthetic Mineral; OASIS® Wound Matrix; ZIMMER Collagen Repair Patch; DeNovo NT Graft marketed by ZIMMER as disclosed in, e.g., U.S. Pat. No. 7,824,711, the disclosure of which is hereby incorporated by reference in its entirety for all purposes; DeNovo ET Engineered Tissue Graft marketed by Zimmer; small intestinal submucosa (SIS) as disclosed in, e.g., U.S. Pat. No. 8,025,896, the disclosure of which is hereby incorporated by reference in its entirety for all purposes; a composite structure such as disclosed in, e.g., U.S. patent application Ser. No. 12/997,611 (published as US 20110091515) the disclosures of which are hereby incorporated in their entireties for all purposes; PROMOGRAN Matrix Wound Dressing; PURAPLY Collagen Dressing; or FIBROCOL PLUS® Collagen Dressing.

### Disposing PDGF Solution in a Biocompatible Matrix

In another aspect, the present invention provides methods for producing compositions for use in the treatment of full thickness burn injuries, for promoting healing and regeneration of impaired or damaged tissue at a site of such injuries, and for promoting revascularization, and angiogenesis in regenerating tissues at such sites. In one embodiment, a method for producing such compositions comprises providing PDGF, optionally present in a pharmaceutically acceptable liquid, providing a biocompatible matrix, and disposing the PDGF or the pharmaceutically acceptable liquid containing PDGF in the biocompatible matrix. PDGF, pharmaceutically acceptable liquids, and biocompatible matrices suitable for combination are consistent with those described hereinabove.

In some embodiments, a PDGF solution can be disposed in a biocompatible matrix by soaking the biocompatible matrix in the PDGF solution. A PDGF solution, in another embodiment, can be disposed in a biocompatible matrix by injecting the biocompatible matrix with the PDGF solution. In some embodiments, injecting a PDGF solution can comprise disposing the PDGF solution in a syringe and expelling the PDGF solution into the biocompatible matrix to saturate the biocompatible matrix.

The biocompatible matrix, according to some embodiments, can be in a predetermined shape, such as a brick or cylinder, prior to receiving a PDGF solution. Subsequent to receiving a PDGF solution, the biocompatible matrix can have a paste or putty form that is flowable, extrudable, and/or injectable. In other embodiments, the biocompatible matrix can already demonstrate a flowable paste or putty form prior to receiving a solution comprising PDGF.

### Compositions Further Comprising Biologically Active Agents

Compositions of the present invention, according to some embodiments, further comprise one or more biologically active agents in addition to PDGF. Biologically active agents that can be incorporated into compositions of the present invention, in addition to PDGF, can comprise: in vitro-prepared tissue, in vitro-prepared dermal tissue, biological components made by or derived thereby, as disclosed above; organic molecules; inorganic materials; proteins; peptides; nucleic acids (e.g., genes, gene fragments, small-insert ribonucleic acids [si-RNAs], gene regulatory sequences, nuclear transcriptional factors and antisense molecules), nucleoproteins; polysaccharides (e.g., heparin); glycoproteins; and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive proteins, growth factors, and vaccines, are disclosed in U.S. patent application Ser. No. 11/159,533 (Publication No: 20060084602). Biologically active compounds that can be incorporated into compositions of the present invention, in some embodiments, include osteoinductive factors such as insulin-like growth factors, fibroblast growth factors, or other PDGFs. In accordance with other embodiments, biologically active compounds that can be incorporated into compositions of the present invention preferably include osteoinductive and osteostimulatory factors such as bone morphogenetic proteins (BMPs), BMP mimetics, calcitonin, calcitonin mimetics, statins, statin derivatives, fibroblast growth factors, insulin-like growth factors, growth differentiating factors, and/or parathyroid hormone. Additional factors for incorporation into compositions of the present invention, in some embodiments, include protease inhibitors, as well as osteoporotic treatments that decrease bone resorption including bisphosphonates, and antibodies to the NF-kB (RANK) ligand.

Standard protocols and regimens for delivery of additional biologically active agents are known in the art. Additional biologically active agents can be introduced into compositions of the present invention in amounts that allow delivery of an appropriate dosage of the agent to injury site or to a particular tissue that is damaged or impaired in or at the site. In most cases, dosages are determined using guidelines known to practitioners and applicable to the particular agent in question. The amount of an additional biologically active agent to be included in a composition of the present invention can depend on such variables as the type and extent of the condition, the overall health status of the particular patient, the formulation of the biologically active agent, release kinetics, and the bioresorbability of the biocompatible matrix. Standard clinical trials may be used to optimize the dose and dosing frequency for any particular additional biologically active agent.

Other exemplary such further biologically active agents include an albumin, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a protease inhibitor, ethylenediaminetetraacetic acid (EDTA), ethylene glycol-bis(beta-aminoethylether)-N, N,N′,N′-tetraacetic acid (EGTA), aprotinin, ε-aminocaproic acid (EACA), a fibroblast growth factor, a keratinocyte growth factor, PDGF-AB, PDGF-AA, PDGF-CC, PDGF, DD, and an osteogenin.

A composition for use in accordance with the disclosed methods may further comprise other materials with PDGF including autologous bone marrow, autologous platelet extracts, allografts, synthetic bone matrix materials, xenografts, and derivatives thereof.

### Use of Compositions Comprising PDGF

The present invention provides for the use of compositions of the present invention for treating full thickness burn injuries, promoting healing or regeneration of damaged or impaired tissue at a site of a full thickness burn injury, promoting vascularization in regenerating tissue at a site of a full thickness bum injury, promoting angiogenesis in regenerating tissue at a site of a full thickness bum injury, promoting angiogenesis in regenerating tissue at a site of a full thickness bum injury, wherein the tissue is selected from the group consisting of bone, periosteum, tendon, muscle, fascia, nerve tissue, vascular tissue, and combinations thereof. The present invention additionally provides for the use of compositions of the present invention in the preparation of a medicament useful for treating full thickness burn injuries, promoting healing or regeneration of damaged or impaired tissue at a site of a full thickness bum injury, promoting vascularization in regenerating tissue at a site of a full thickness burn injury, promoting angiogenesis in regenerating tissue at a site of a full thickness burn injury, promoting angiogenesis in regenerating tissue at a site of a full thickness burn injury, wherein the tissue is selected from the group consisting of bone, periosteum, tendon, muscle, fascia, nerve tissue, vascular tissue, and combinations thereof.

### Kits

In another aspect, the present invention provides a kit comprising a solution comprising PDGF in a first container and a second container comprising a biocompatible matrix. In some embodiments, the solution comprises a predetermined concentration of PDGF. The concentration of PDGF, in some embodiments, can be predetermined according to the nature of the bum injury being treated. Moreover, the amount of biocompatible matrix provided by a kit can be dependent on the nature of the bum injury being treated. A biocompatible matrix that may be included in the kit may comprise one or more of the matrix components described above. In one embodiment the bone comprises a type I collagen patch or pad as described herein. A syringe, in some embodiments, can facilitate disposition of the PDGF solution in the biocompatible matrix for application at burn injury site. The kit may also contain instructions for use.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

### Treating a Full Thickness Burn Injury Site in a Patient

At the time of application of the composition comprising the biocompatible matrix and the effective amount of rhPDGF-BB, the patient will be taken to a procedural area with environmental controls and full intensive care monitoring. The patient will be sedated as necessary and in a fashion that is consistent with standards of care for full thickness burn injuries. The site of the burn injury is cleaned and debrided of impaired tissue, including burned, damaged and/or loosely adherent necrotic tissue. Following debridement, exposed bone is decorticated to remove damaged and/or necrotic periosteum. Intramarrow bone penetrations are then performed to produce bleeding bone and facilitate egress of migrating stromal cells, mesenchymal stem cells and other marrow components from the marrow into the injury site. A sterile 0.3 mg/ml solution of rhPDGF-BB in sodium acetate buffer is used to saturate a collagen matrix which is then applied onto exposed bone, muscle and surrounding tissue. One or more of an antimicrobial agent, such as povidone-iodine, an antibiotic such as mafenide-acetate, an antiseptic such as chlorhexadine, and the like, as described above, is optionally applied onto exposed tissues with the burn site either separately or as a component of the biocompatible matrix/PDGF composition. It is estimated that up to 10 cubic centimeters of the rhPDGF-BB solution will initially be required to treat a surface area of approximately 27 centimeters squared (cm³). One or more matrix pads may be required in order to cover the approximately 27 cm³ surface area to be treated. Approximately one to two times per week for approximately 12 weeks, either: subsequent effective amounts of PDGF solution will be applied to the matrix at the injury site; or subsequent compositions comprising the matrix into which the PDGF has been disposed therein will be applied at the injury site., after which the patient's progress will be assessed. If significant healing of the site has occurred, therapy is continued for another twelve weeks, or until a vascularized bed of healthy tissue has formed that is sufficient to support a graft. It is anticipated that the amount of rhPDGF-BB and collagen applied in each treatment will diminish over time as the injury heals.

Following application of the rhPDGF-BB soaked collagen matrix, the site will be dressed in fashion standard for full thickness burn injuries. This will include a nonadherent, fine meshed dressing, coarse cotton gauze wrap dressings saturated with the topical antimicrobial Mafenide Acetate solution, and finally an elastic bandage. The PDGF solution is optionally applied to this dressing. A skin graft or skin flap will be applied to the injury site once the

The patient is monitored daily for any signs of adverse events related to the treatment. Local and systemic changes are noted in the patient's medical records.

### EXAMPLE 2

### Treating a Full Thickness Burn Injury Site in a Patient

At the time of application of the composition comprising the biocompatible matrix and the effective amount of rhPDGF-BB, the patient will be taken to a procedural area with environmental controls and full intensive care monitoring. The patient will be sedated as necessary and in a fashion that is consistent with standards of care for full thickness burn injuries. The site of the burn injury, including impaired or damaged tissue, is cleaned. A sterile 0.3 mg/ml solution of rhPDGF-BB in sodium acetate buffer is used to saturate a collagen matrix which is then applied onto exposed bone, periosteum, tendon, muscle, nerve tissue, and/or fascia, and other surrounding tissue that appears to be impaired or damaged at or within the site of the burn injury. One or more of an antimicrobial agent, such as povidone-iodine, an antibiotic such as mafenide-acetate, an antiseptic such as chlorhexadine, and the like, as described above, is optionally applied onto exposed tissues with the burn site either separately or as a component of the biocompatible matrix/PDGF composition.

It is estimated that up to 10 cubic centimeters of the rhPDGF-BB solution will initially be required to treat a surface area of approximately 27 centimeters squared (cm³). One or more matrix pads may be required in order to cover the approximately 27 cm³ surface area to be treated. Approximately one to two times per week for approximately 12 weeks, either: subsequent effective amounts of PDGF solution will be applied to the matrix at the injury site; or subsequent compositions comprising the matrix into which the PDGF has been disposed therein will be applied at the injury site., after which the patient's progress will be assessed. If significant healing of the site has occurred, therapy is continued for another twelve weeks, or until a vascularized bed of healthy tissue has formed that is sufficient to support a graft. It is anticipated that the amount of rhPDGF-BB and collagen applied in each treatment will diminish over time as the injury heals.

Following application of the rhPDGF-BB soaked collagen matrix, the site will be dressed in fashion standard for full thickness burn injuries. This will include a nonadherent, fine meshed dressing, coarse cotton gauze wrap dressings saturated with the topical antimicrobial Mafenide Acetate solution and/or optionally another antimicrobial agent, such as antibiotic, an antibacterial agent, an iodine-containing agent, a peroxide-containing agent, a silver-containing agent, iodine, iodide ion, hydrogen peroxide, peroxide ion, or silver ion, and the like, and finally an elastic bandage. The PDGF solution is optionally applied to this dressing. A skin graft or skin flap will be applied to the injury site once the

The patient is monitored daily for any signs of adverse events related to the treatment. Local and systemic changes are noted in the patient's medical records.

We claim:

1. A method of treating, promoting healing at and/or promoting vascularization at a site of a burn injury in a subject in need thereof, the method comprising:
a) applying a composition comprising a biocompatible matrix and an effective amount of platelet-derived growth factor (PDGF) disposed therein to the site.

2. The method according to claim 1, wherein the site comprises tissue selected from the group consisting of bone, periosteum, tendon, muscle, fascia, nerve tissue, vascular tissue, and combinations thereof.

3. The method according to claim 1, wherein the burn injury is a full thickness burn injury.

4. The method according to claim 1, wherein the method further comprises debriding at least a portion of a site of impaired, damaged, or necrotic tissue prior to performing step (a).

5. The method according to claim 1, wherein the method further comprises decorticating at least a portion of impaired, damaged or necrotic bone at the site.

6. The method according to claim 1, further comprising performing at least one intramarrow bone penetration at the site.

7. The method according to claim 1, wherein the burn injury comprises a third degree burn, a fourth degree burn, a chemical burn, a thermal burn, or an electrical burn.

8. The method according to claim 1, wherein the method further comprises: disposing in the biocompatible matrix at least one subsequent effective amount of PDGF

9. The method according to claim 1, wherein the method further comprises applying at least one subsequent composition comprising a biocompatible matrix and an effective amount of PDGF disposed therein to the site.

10. The method according to claim 1, wherein the biocompatible matrix is bioresorbable.

11. The method according to claim 1, wherein the biocompatible matrix comprises proteins, polysaccharides, nucleic acids, carbohydrates, synthetic polymers, or mixtures thereof.\

12. The method according to claim 1, wherein the biocompatible matrix is porous.

13. The method according to claim 1, wherein the biocompatible matrix comprises a mesh, a gauze, a sponge, a monophasic plug, a biphasic plug, a paste, a putty, a wrap, a bandage, a patch, or a pad.

14. The method according to claim 1, wherein the biocompatible matrix comprises a polyurethane, a siloxane, a polysiloxane, a collagen, a Type I collagen, a Type II collagen, a Type III collagen, a Type IV collagen, a Type V collagen, a Type VI collagen, a type VII collagen, a Type VIII collagen, a glycosaminoglycan, oxidized regenerated cellulose (ORC), an ORC:collagen composite, an alginate, an alginate:collagen composite, an ethylene diamine tetraacetic acid (EDTA),a poly(lactic-co-glycolitic acid (PLGA), a carboxymethylcellulose, a granulated collagen-glycosaminoglycan composite, methylcellulose, hydroxypropyl methylcellulose, or hydroxyethyl cellulose alginic acid, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), polyvinyl alcohol), poly(vinylpyrrolidone), poly(ethyloxazoline), poly(ethylene oxide)-co-poly(propylene oxide) block copolymers, poly(ethylene terephthalate)polyamidearabic gum, guar gum, xantham gum, gelatin, chitin, chitosan, chitosan acetate, chitosan lactate, chondroitin sulfate, N,O-carboxymethyl chitosan, a dextran, fibrin glue, glycerol, hyaluronic acid, sodium hyaluronate, a cellulose, a glucosamine, a proteoglycan, a starch, lactic acid, a pluronic, sodium glycerophosphate, glycogen, a keratin, a silk, an in vitro-prepared tissue, an in vitro-prepared dermal tissue, a biological component made by an in vitro-prepared tissue, a biological component derived from an in vitro-derived tissue, one or more composites thereof, one or more mixtures thereof, or one or more combinations thereof.

15. The method according to claim 1, wherein the biocompatible matrix comprises a collagen.

16. Then method according to claim 1, wherein the biocompatible matrix comprises Type I collagen, Type II collagen, Type III collagen, bovine collagen, human collagen, porcine collagen, equine collagen, avian collagen, or combinations thereof.

17. The method according to claim 1, wherein the method further comprises applying a dressing, a skin flap, or a graft at the site after performing step a).

18. The method according to claim 17, wherein the dressing, skin flap, or graft comprises a cadaver-derived skin graft, an animal skin graft, a skin autograft, a skin allograft, a synthetic skin substitute, a dermal substitute, fibroblast-derived temporary skin substitute, an artificial skin, a reconstructive tissue matrix, an acellular tissue matrix, an acellular dermal matrix (ADM), a processed human tissue graft, a reconstructive tissue matrix, a bioengineered cell construct, a bioengineered collagen matrix, an engineered tissue graft, a cellular and collagen construct, a collagen dressing, a human bi-layered bio-engineered skin, or a polyglactin mesh scaffold.

19. The method according to claim 17, wherein an effective amount of PDGF is disposed in the dressing, the skin flap, or the graft.

20. The method according to claim 1, wherein an antimicrobial agent is disposed in the biocompatible matrix.

21. The method according to claim 20, wherein the antimicrobial agent comprises an antibiotic, an anticeptic, an antibacterial agent, an iodine-containing agent, a peroxide-containing agent, a silver-containing agent, iodine, povidone-iodine, an iodide ion-containing agent, hydrogen peroxide, a peroxide ion-containing agent, or a silver ion-containing agent, or chlorhexadine.

22. The method according to claim 20, wherein the anti-microbial agent is selected from the group consisting of: mafenide-acetate, penicillin, ampicillin, penicillin G, clindamycin (Cleocin), Ceftriaxone (Rocephin), erythromycin, gentamicin (Garamycin), clindamycin (Cleocin), metronidazole (Flagyl), Ampicillin-sulbactam (Unasyn), ticarcillin-clavulanate potassium (Timentin), piperacillin-tazobactam (Zosyn), nafcillin (Unipen), Imipenem-cilastatin (Primaxin), a β-lactam, a β-lactamase inhibitor, antipseudomonal cephalosporin, ceftazidime (Fortaz), clindamycin, metronidazole, Vancomycin (Vancocin), an aminoglycoside, aztreonam (Azactam), amphotericin B (Abelcet), a third-generation cephalosporin, and combinations thereof.

23. The method according to claim 1, wherein at least one further biological agent is administered to the subject.

24. The method according to claim 23 wherein the at least one further biologically active agent is selected the group consisting of: an albumin, a growth factor, a cytokine, a VEGF, a PDGF, a BMP, insulin-like growth factor I (IGF-I), an insulin-like growth factor II (IGF-II), a transforming growth factor-β1 (TGF-β1), a transforming growth factor-β2 (TGF-β2), a transforming growth factor-α (TGF-α), a bone morphogenetic protein (BMP), a fibroblast growth factor (FGF), an epidermal growth factor (EGF), a keratinocyte growth factor, PDGF-AB, PDGF-AA, PDGF-CC, PDGF-DD, an osteogenin, a protease inhibitor, a metalloproteinase inhibitor, a metalloproteinase-3 inhibitor, ethylenediaminetetraacetic acid (EDTA), ethylene glycol-bis(beta-aminoethylether)-N, N,N′,N′-tetraacetic acid (EGTA), aprotinin, and ε-aminocaproic acid (EACA).

25. The method according to claim 1, wherein the PDGF is obtained from a natural source or a recombinant source.

26. The method according to claim 25, wherein the natural source comprises blood, platelets, serum, platelet concentrate, platelet-rich plasma (PRP), platelet-poor plasma (PPP), platelet-free plasma, plasma, fresh frozen plasma (FFP), or bone marrow.

27. The method according to claim 12, wherein the natural source is platelet-rich plasma (PRP).

28. The method according to claim 1, wherein the PDGF is PDGF-AA, PDGF-BB, PDGF-AB, PDGF-CC, PDGF-DD, or a mixture or a derivative thereof.

29. The method according to claim 1, wherein the PDGF is in a pharmaceutically acceptable liquid.

30. The method according to claim 1, wherein the PDGF is at a concentration of about 0.01 mg/ml to about 10 mg/ml.

31. The method according to claim 1, wherein the PDGF comprises a PDGF-BB. The method according to claim 1, wherein the PDGF comprises a recombinant human (rh) PDGF-BB.

32. A kit for use in the method of claim 1, wherein the kit comprises a biocompatible matrix and an effective amount of PDGF.
